(19) United States
(12) Patent Application Publication
(10) Pub. No.: US 2023/0105693 A1
(43) Pub. Date: Apr. 6, 2023

(54) ANTIMICROBIAL COMPOSITIONS

(71) Applicant: Firmenich SA, Satigny (CH)

(72) Inventors: Qiumin MA, Shanghai (CN); Yang HUANG, Shanghai (CN); Wolfgang FIEBER, Satigny (CH)

(21) Appl. No.: 17/799,856

(22) PCT Filed: Feb. 18, 2021
(86) PCT No.: PCT/EP2021/054074

§ 371 Date: Aug. 15, 2022

(30) Foreign Application Priority Data
Feb. 21, 2020 (WO) PCT/CN2020/076166
Jun. 4, 2020 (EP) 20178324.8

(51) Int. Cl. A61K 8/34; A61K 8/40; A61Q 17/00; A61Q 15/00; A61Q 13/00; A61Q 19/10; A01N 31/08; A01N 31/02; A01N 35/02; A01P 1/00; A61K 8/33; C11D 3/48; C11D 3/50

(52) U.S. Cl. CPC A61K 8/347; A01N 31/02; A01N 31/08; A01N 35/02; A01P 1/00; A61K 8/33; A61K 8/34; A61K 8/40; A61Q 13/00; A61Q 15/00; A61Q 17/005; A61Q 19/10; C11D 3/48; C11D 3/50

(57) ABSTRACT

The present invention is related to methods and compositions that can be applied to personal cleaning, oral care, deodorant and hard surface cleaning applications, including liquid soap, foaming soap, liquid dishwashing detergent, shower gel, shampoo, emulsified deodorant, mouthwash, toothpaste and facial cleanser in order to reduce or eliminate microbes on a surface or body part.

### FIELD OF THE INVENTION

The present invention is related to methods and compositions for human or animal body surface and hair cleaning applications, soft and porous substrates like fabric or hard surfaces cleaning applications, deodorant, oral care, or air care applications in order to reduce or eliminate microbes.

### BACKGROUND

Perfume ingredients may have antimicrobial activity. However, the antimicrobial activity of a perfume ingredient can be largely impacted by the application bases, specifically, the surfactant bases, e.g., liquid soaps or shampoo. In these bases, perfume ingredients are incorporated into surfactant micelles and the bioavailability of the perfume ingredients is significantly reduced. Therefore, much higher concentrations of perfume ingredients are needed, but are not acceptable in terms of sensory affect, cost, and solubility issues. These obstacles are overcome by the compositions, methods, and uses of the present invention.

### SUMMARY OF THE INVENTION

The present invention encompasses methods and antimicrobial compositions comprising a perfume ingredient and a surfactant, wherein the perfume ingredient has a log P of less than or equal to 3.5, and a bactericidal effect of a 5.5 log reduction in an ethanol solution at a concentration of less than or equal to 0.5% in ethanol solution.

In an aspect of the invention, the perfume ingredient has a bactericidal effect of a 5.5 log reduction in an ethanol solution at a concentration of less than or equal to 0.2% in an ethanol solution. In a further aspect, the perfume ingredient has a bactericidal effect of a 5.5 log reduction at a concentration of less than or equal to 0.1% in an ethanol solution.

A perfume ingredient the present invention preferably may have a log P of less than or equal to 3.0.

A perfume ingredient of the present invention may have an equivalent alkane carbon number of less than or equal to -10. Further, according to the present invention, the perfume ingredient may have an equivalent alkane carbon number of less than or equal to -20.

In aspects of the present invention, an antimicrobial composition includes a mixture of 2-ethyl-1-hexanol, 5-isopropyl-2-methylphenol, (E)-3,7-dimethyl-2,6-octadien-1-ol, and 2-isopropyl-5-methylphenol at various ratios. In other aspects, the antimicrobial composition includes a mixture of (E)-3-phenyl-2-propenal; (Z)-3,7-dimethyl-2,6-octadien-1-ol; 1,8-p-methadien-7-ol; and 3-methyl-5-phenyl-1-pentanol at various ratios. In other aspects, the antimicrobial composition includes a mixture of 2-ethyl-1-hexanol; (Z)-3,7-dimethyl-2,6-octadien-1-ol; 3-mthyl-5-phenyl-1-pentanol; and 5-methyl-3-heptanone oxime at various ratios. Further, according to the present invention, an antimicrobial composition may include a mixture of 2-ethyl-1-hexanol, 5-isopropyl-2-methylphenol, (E)-3,7-dimethyl-2,6-octadien-1-ol, and 2-isopropyl-5-methylphenol in a 5:3:8:4 ratio. In further aspect, the antimicrobial composition of may include a mixture of (E)-3-phenyl-2-propenal; (Z)-3,7-dimethyl-2,6-octadien-1-ol; 1,8-p-methadien-7-ol; and 3-methyl-5-phenyl-1-pentanol in a 5:5:4:6 ratio. In further aspects, the antimicrobial composition may include a mixture of 2-ethyl-1-hexanol; (Z)-3,7-dimethyl-2,6-octadien-1-ol; 3-methyl-5-phenyl-1-pentanol; and 5-methyl-3-heptanone oxime in a 3:3:3:1 ratio. In further aspects, the antimicrobial composition may include a mixture of 5-isopropyl-2-methylphenol, (E)-3,7-dimethyl-2,6-octadien-1-ol, and 2-isopropyl-5-methylphenol in a 3:8:4 ratio.

In further aspect, the antimicrobial composition of may include a mixture of (E)-3-phenyl-2-propenal, 5-isopropyl-2-methylphenol, 2-ethyl-1-hexanol, (E)-3,7-dimethyl-2,6-octadien-1-ol, (Z)-3,7-dimethyl-2,6-octadien-1-ol, 1,8-p-menthadien-7-ol, 3-methyl-5-phenyl-1-pentanol, 5-methyl-3-heptanone oxime and 2-lsopropyl-5-methylphenol.

A perfume ingredient in a composition of the invention may be present in an amount effective to provide an antimicrobial effect.

In aspects of the present invention, the perfume ingredient is at least 0.001 % (w/v) of the antimicrobial composition.

A surfactant may be 0.1% to 30% (w/w) of the antimicrobial composition. In certain aspects, the surfactant is 0.1% to 20% (w/w) of the antimicrobial composition. In further aspects, the surfactant is 0.1% to 10% (w/w) of the antimicrobial composition. In a further aspect the surfactant is 9.1% to 30% (w/w) of the antimicrobial composition. In a further aspect the surfactant is 9.1% to 20% (w/w) of the antimicrobial composition. In a further aspect the surfactant is 9.1% to 10% (w/w) of the antimicrobial composition.

A surfactant of the present invention is may be an anionic surfactant, a non-ionic surfactant, an amphoteric surfactant or a combination thereof.

The anionic surfactant may be, for example, sodium lauryl ether sulfate, and the amphoteric surfactant may be cocamidopropyl betaine. The non-ionic surfactant may be an alkyl polyglycoside. A non-ionic surfactant according to the present invention may be coco-glucoside.

In aspects of the present invention, the antimicrobial composition may further include a hydrotrope. In further aspects, the antimicrobial composition comprises an additional agent active against gram-positive or gram-negative bacteria. In other aspects, the antimicrobial composition may further comprise a chelating agent selected from the group consisting of EDTA, and CDTA, and a combination thereof.

A perfume ingredient according to the present invention may be 1-phenylethyl acetate, (2E)-2-methyl-3-phenyl-2-propenal, (2E,6Z)-2,6-nonadien-1-ol, 5-isopropyl-2-methylphenol, ethyl (E)-3-phenyl-2-propenoate, 3,7-dimethyl-2,6-octadienal, 3-[4-methyl-3-cyclohexen-1-yl]-1-butanol, 4-decanolide, 2-methoxy-4-propylphenol, (E)-3,7-dimethyl-2,6-octadien-1-ol, 2,4,6-trimethyl-3-cyclohexene-1-methanol, (2E)-2-methyl-3-(4-methylphenyl)-2-propen-1-ol, 2,5-dimethyl-2-indanmethanol, 2,2-dimethyl-3-[3-methyl-2,4-pentadien-1-yl]oxirane, 2-ethyl-1-hexanol, (Z)-3,7-dimethyl-2,6-octadien-1-ol, (Z)-6-nonen-1-ol, 1,8-p-menthadien-7-ol, 3-methyl-5-phenyl-1-pentanol, 2,6,6-trimethyl-1,3-cyclohexadiene-1-carbaldehyde, 2-isopropyl-5-methylphenol, 3-propylphenol, ethyl 2-hydroxybenzoate, 4-isopropyl-1-benzenemethanol, 7-propyl-2H,4H-1,5-benzodioxepin-3-one, 2-methoxy-4-(2-propen-1-yl)phenol, 3-(1,3-benzodioxol-5-yl)-2-methylpropanal, indole, 2-methoxy-4-[(1E)-1-propen-1-yl]phenol, 3-methyl-2-[(2Z)-2-penten-1-yl]-2-cyclopenten-1-one, (E)-2-hexenyl acetate, 4-(2-methyl-2-propanyl)cyclohexanone, ethyl phenylacetate, 5-methyl-3-heptanone oxime, 2-(4-methylcyclohex-3-enyl)propan-2-ol, (E)-3-phenyl-2-propenenitrile, 2-phenyl-1-propanol, (E)-3-phenyl-2-propenal, methyl 2-aminobenzoate, 4-nonanolide, 6-pentyltetrahydro-2H-pyran-2-one, 2-methyl-4-phenyl-2-butanol, 2-methyl-1-phenyl-2-propanol, 2-phenylethyl formate, (E)-2-hexen-1-ol, 4-methylphenol, 4,4a,6,7,8,8a-hexahydro-1,4-methanonaphthalen-5(1H)-one, 1-oxo-1-(2-propanyloxy)-2-propanyl 2,2-dimethylpropanoate, 2,4,6-trimethyl-3-cyclohexene-1-carbaldehyde, 3,5,6-trimethyl-3-cyclohexene-1-carbaldehyde, 3-(4-hydroxy-4-methylpentyl)-3-cyclohexene-1-carbaldehyde, 4-(4-hydroxy-4-methylpentyl)-3-cyclohexene-1-carbaldehyde, (2E)-3-phenyl-2-propen-1-ol, 2-furanmethanethiol, (Z)-3-hexenyl formate, 2-phenylethyl acetate, 1-octanol, 1,3,3-trimethylbicyclo[2.2.1]heptan-2-ol, (Z)-2-nonenal, methyl 4-methoxybenzoate, 1,3-nonanediyl diacetate, tetrahydro-3-pentyl-4(2H)-pyranyl acetate, 1-(5-propyl-1,3-benzodioxol-2-yl)ethenone, ethyl benzoate, 1,7,7-trimethylbicyclo[2.2.1]heptan-2-ol, benzyl butanoate, 1-butoxycarbonylethyl butanoate, ethyl hexanoate, 4,8-cyclododecadien-1-one, methyl (2E)-2-methyl-2-hexenoate, methyl (E)-3-phenyl-2-propenoate, (Z)-3-hexenyl acetate, 3,5,5-trimethyl-1-hexanol, methyl cyclopentylideneacetate, (Z)-4-decenal, 2,6-dimethyl-7-octen-4-one, 2,6-dimethyl-4-heptanol, 3-(2,2-dimethylpropyl)pyridine, (Z)-7-decen-4-olide, clove oil, isobutyl isobutyrate, 2,4-dimethyl-4,4a,5,9b-tetrahydroindeno[1,2-d][1,3]dioxine, 1,2-dimethoxy-4-[1-propen-1-yl]benzene, 1-(3-methyl-1-benzofuran-2-yl)ethenone, (2E,6Z)-2,6-nonadienal, 3-methylbutyl propionate, 2-methylbutyl propionate, 2-isobutyl-3-methoxypyrazine, 2-isobutyl-6-methoxypyrazine, methyl 2-hydroxybenzoate, 3-methylindole, 1-methoxy-3-hexanethiol, 8-mercapto-3-p-menthanone, 1,3,3-trimethylbicyclo[2.2.1]heptan-2-one, 1-isopropyl-4-methylbicyclo[3.1.0]hexan-3-one, 3-phenylbutanal, 6-hexyltetrahydro-2H-pyran-2-one, 7-isopropyl-2H,4H-1,5-benzodioxepin-3-one, spearmint oil or a combination thereof.

Further, the perfume ingredient may be 1-phenylethyl acetate, (2E)-2-methyl-3-phenyl-2-propenal, (2E,6Z)-2,6-nonadien-1-ol, 5-isopropyl-2-methylphenol, ethyl (E)-3-phenyl-2-propenoate, 3,7-dimethyl-2,6-octadienal, 2-methoxy-4-propylphenol, (E)-3,7-dimethyl-2,6-octadien-1-ol, (2E)-2-methyl-3-(4-methylphenyl)-2-propen-1-ol, 2,2-dimethyl-3-[3-methyl-2,4-pentadien-1-yl]oxirane, 2-ethyl-1-hexanol, (Z)-3,7-dimethyl-2,6-octadien-1-ol, 1,8-p-menthadien-7-ol, 3-methyl-5-phenyl-1-pentanol, 2,6,6-trimethyl-1,3-cyclohexadiene-1-carbaldehyde, 2-isopropyl-5-methylphenol, 3-propylphenol, 4-isopropyl-1-benzenemethanol, 7-propyl-2H,4H-1,5-benzodioxepin-3-one, 2-methoxy-4-(2-propen-1-yl)phenol, 3-(1,3-benzodioxol-5-yl)-2-methylpropanal, indole, 2-methoxy-4-[(1E)-1-propen-1-yl]phenol, 3-methyl-2-[(2Z)-2-penten-1-yl]-2-cyclopenten-1-one, (E)-2-hexenyl acetate, 4-(2-methyl-2-propanyl)cyclohexanone, ethyl phenylacetate, 5-methyl-3-heptanone oxime, 2-(4-methylcyclohex-3-enyl)propan-2-ol, (E)-3-phenyl-2-propenenitrile, 2-phenyl-1-propanol, (E)-3-phenyl-2-propenal, methyl 2-aminobenzoate, 4-nonanolide, 6-pentyltetrahydro-2H-pyran-2-one, 2-methyl-4-phenyl-2-butanol, 2-methyl-1-phenyl-2-propanol, 2-phenylethyl formate, (E)-2-hexen-1-ol, 4-methylphenol, 4,4a,6,7,8,8a-hexahydro-1,4-methanonaphthalen-5(1H)-one, 1-oxo-1-(2-propanyloxy)-2-propanyl 2,2-dimethylpropanoate, 3-(4-hydroxy-4-methylpentyl)-3-cyclohexene-1-carbaldehyde, 4-(4-hydroxy-4-methylpentyl)-3-cyclohexene-1-carbaldehyde, (2E)-3-phenyl-2-propen-1-ol, 2-furanmethanethiol, (Z)-3-hexenyl formate, 2-phenylethyl acetate, 1-octanol, 1,3,3-trimethylbicyclo[2.2.1]heptan-2-ol, methyl 4-methoxybenzoate, 1,3-nonanediyl diacetate, tetrahydro-3-pentyl-4(2H)-pyranyl acetate, 1-(5-propyl-1,3-benzodioxol-2-yl)ethenone, ethyl benzoate, 1,7,7-trimethylbicyclo[2.2.1]heptan-2-ol, benzyl butanoate, 1-butoxycarbonylethyl butanoate, methyl (2E)-2-methyl-2-hexenoate, methyl (E)-3-phenyl-2-propenoate, (Z)-3-hexenyl acetate, 3,5,5-trimethyl-1-hexanol, methyl cyclopentylideneacetate, (Z)-4-decenal, 2,6-dimethyl-4-heptanol, (Z)-7-decen-4-olide, isobutyl isobutyrate, 2,4-dimethyl-4,4a,5,9b-tetrahydroindeno[1,2-d][1,3]dioxine, 1,2-dimethoxy-4-[1-propen-1-yl]benzene, 1-(3-methyl-1-benzofuran-2-yl)ethenone, (2E,6Z)-2,6-nonadienal, 3-methylbutyl propionate, 2-methylbutyl propionate, 2-isobutyl-3-methoxypyrazine, 2-isobutyl-6-methoxypyrazine, methyl 2-hydroxybenzoate, 3-methylindole, 8-mercapto-3-p-menthanone, 4,4a,6,7,8,8a-hexahydro-1,4-methanonaphthalen-5(1H)-one, 1,3,3-trimethylbicyclo[2.2.1]heptan-2-one, 1-isopropyl-4-methylbicyclo[3.1.0]hexan-3-one, 3-phenylbutanal, 6-hexyltetrahydro-2H-pyran-2-one, 7-isopropyl-2H,4H-1,5-benzodioxepin-3-one, clove oil, spearmint oil or a combination thereof.

In an aspect of the present invention, the perfume ingredient is (2E)-2-methyl-3-phenyl-2-propenal, (2E,6Z)-2,6-nonadien-1-ol, 5-isopropyl-2-methylphenol, 2-methoxy-4-propylphenol, (E)-3,7-dimethyl-2,6-octadien-1-ol, (2E)-2-methyl-3-(4-methylphenyl)-2-propen-1-ol, 2-ethyl-1-hexanol, (Z)-3,7-dimethyl-2,6-octadien-1-ol, 1,8-p-menthadien-7-ol, 3-methyl-5-phenyl-1-pentanol, 2-isopropyl-5-methylphenol, 3-propylphenol, 4-isopropyl-1-benzenemethanol, 7-propyl-2H,4H-1,5-benzodioxepin-3-one, 2-methoxy-4-(2-propen-1-yl)phenol, 3-(1,3-benzodioxol-5-yl)-2-methylpropanal, indole, 2-methoxy-4-[(1E)-1-propen-1-yl]phenol, 5-methyl-3-heptanone oxime, 2-(4-methylcyclohex-3-enyl)propan-2-ol, (E)-3-phenyl-2-propenenitrile, 2-phenyl-1-propanol, (E)-3-phenyl-2-propenal, methyl 2-aminobenzoate, 4-nonanolide, 6-pentyltetrahydro-2H-pyran-2-one, 2-methyl-4-phenyl-2-butanol, 2-methyl-1-phenyl-2-propanol, (E)-2-hexen-1-ol, 4-methylphenol, 1-oxo-1-(2-propanyloxy)-2-propanyl 2,2-dimethylpropanoate, 3-(4-hydroxy-4-methylpentyl)-3-cyclohexene-1-carbaldehyde, 4-(4-hydroxy-4-methylpentyl)-3-cyclohexene-1-carbaldehyde, (2E)-3-phenyl-2-propen-1-ol, 2-furanmethanethiol, (Z)-3-hexenyl formate, 1-octanol, 1,3,3-trimethylbicyclo[2.2.1]heptan-2-ol, 1-(5-propyl-1,3-benzodioxol-2-yl)ethenone, 1,7,7-trimethylbicyclo[2.2.1]heptan-2-ol, 7-isopropyl-2H,4H-1,5-benzodioxepin-3-one, 3,5,5-trimethyl-1-hexanol, 2,6-dimethyl-4-heptanol, 3-methylindole, 6-hexyltetrahydro-2H-pyran-2-one, clove oil, spearmint oil or a combination thereof.

In a further aspect, the perfume ingredient is (2E,6Z)-2,6-nonadien-1-ol, 5-isopropyl-2-methylphenol, 2-methoxy-4-propylphenol, (E)-3,7-dimethyl-2,6-octadien-1-ol, (2E)-2-methyl-3-(4-methylphenyl)-2-propen-1-ol, 2-ethyl-1-hexanol, (Z)-3,7-dimethyl-2,6-octadien-1-ol, 1,8-p-menthadien-7-ol, 3-methyl-5-phenyl-1-pentanol, 2-isopropyl-5-methylphenol, 3-propylphenol, 4-isopropyl-1-benzenemethanol, 7-propyl-2H,4H-1,5-benzodioxepin-3-one, 2-methoxy-4-(2-propen-1-yl)phenol, indole, 2-methoxy-4-[(1E)-1-propen-1-yl]phenol, 2-phenyl-1-propanol, methyl 2-aminobenzoate, 2-methyl-4-phenyl-2-butanol, (E)-2-hexen-1-ol, 4-methylphenol, 1-oxo-1-(2-propanyloxy)-2-propanyl 2,2-dimethylpropanoate, 3-(4-hydroxy-4-methylpentyl)-3-cyclohexene-1-carbaldehyde, 4-(4-hydroxy-4-methylpentyl)-3-cyclohexene-1-carbaldehyde, (2E)-3-phenyl-2-propen-1-ol , 2-furanmethanethiol, (Z)-3-hexenyl formate, 1-octanol, 1-(5-propyl-1,3-benzodioxol-2-yl)ethenone, 3,5,5-trimethyl-1-hexanol, 3-methylindole, 7-isopropyl-2H,4H-1,5-benzodioxepin-3-one, clove oil or a combination thereof.

In aspects of the present invention, the perfume ingredient is (2E,6Z)-2,6-nonadien-1-ol, 5-isopropyl-2-methylphenol, 2-methoxy-4-propylphenol, (E)-3,7-dimethyl-2,6-octadien-1-ol, (2E)-2-methyl-3-(4-methylphenyl)-2-propen-1-ol, 2-ethyl-1-hexanol, (Z)-3,7-dimethyl-2,6-octadien-1-ol, 1,8-p-menthadien-7-ol, 3-methyl-5-phenyl-1-pentanol, 2-isopropyl-5-methylphenol, 3-propylphenol, 4-isopropyl-1-benzenemethanol, 7-propyl-2H,4H-1,5-benzodioxepin-3-one, 2-methoxy-4-(2-propen-1-yl)phenol, indole, 2-methoxy-4-[(1E)-1-propen-1-yl]phenol, 2-phenyl-1-propanol, 1-oxo-1-(2-propanyloxy)-2-propanyl 2,2-dimethylpropanoate , 2-furanmethanethiol, (Z)-3-hexenyl formate, 1-octanol, 1-(5-propyl-1,3-benzodioxol-2-yl)ethenone, 7-isopropyl-2H,4H-1,5-benzodioxepin-3-one, 3,5,5-trimethyl-1-hexanol, 3-methylindole, clove oil or a combination thereof.

The perfume ingredient according to the present invention may be (2E,6Z)-2,6-nonadien-1-ol, 5-isopropyl-2-methylphenol, 2-methoxy-4-propylphenol, (E)-3,7-dimethyl-2,6-octadien-1-ol, (2E)-2-methyl-3-(4-methylphenyl)-2-propen-1-ol, 2-ethyl-1-hexanol, (Z)-3,7-dimethyl-2,6-octadien-1-ol, 1,8-p-menthadien-7-ol, 3-methyl-5-phenyl-1-pentanol, 2-isopropyl-5-methylphenol, 3-propylphenol , (E)-3-phenyl-2-propenal, 5-methyl-3-heptanone oxime , 2-furanmethanethiol, (Z)-3-hexenyl formate, 1-octanol, 1-(5-propyl-1,3-benzodioxol-2-yl)ethenone, 3,5,5-trimethyl-1-hexanol, 2-methoxy-4-[(1E)-1-propen-1-yl]phenol, 3-methylindole or a combination thereof.

As can be seen in the accompanying examples, a mixture of perfume ingredients labeled Mixture C showed a surprising synergistic antimicrobial effect,; i.e. an effect which is superior to the simple sum or addition of the antimicrobial effect expected when the ingredients of the composition would have been admixed in the desired concentration. In other words, in such cases, antibacterial activity of the combination of combined ingredients is greater than the sum of activities of the individual ingredients.

Mixture C comprises 5-isopropyl-2-methylphenol, 2-ethyl-1-hexanol, (E)-3,7-dimethyl-2,6-octadien-1-ol, 2-lsopropyl-5-methylphenol, or a combination thereof.

In a further embodiment, the perfume ingredient is 2-ethyl-1-hexanol, (Z)-3,7-dimethyl-2,6-octadien-1-ol, 3-methyl-5-phenyl-1-pentanol, 5-methyl-3-heptanone oxime or a combination thereof. In another embodiment, the perfume ingredient is (E)-3-phenyl-2-propenal, (Z)-3,7-dimethyl-2,6-octadien-1-ol, 1,8-p-menthadien-7-ol, 3-methyl-5-phenyl-1-pentanol. In another embodiment, the perfume ingredient is 5-isopropyl-2-methylphenol, (E)-3,7-dimethyl-2,6-octadien-1-ol, 2-lsopropyl-5-methylphenol.

In an embodiment of the present invention, the perfume ingredient is (2E)-2-methyl-3-phenyl-2-propenal, (2E,6Z)-2,6-nonadien-1-ol, 5-isopropyl-2-methylphenol, (E)-3,7-dimethyl-2,6-octadien-1-ol 2-ethyl-1-hexanol, (Z)-3,7-dimethyl-2,6-octadien-1-ol, (-)-(5)-1,8-P-menthadiene-7-ol, (+-)-3-methyl-5-phenyl-1-pentanol, 2-isopropyl-5-methylphenol, 3-propylphenol, 5-methyl-3-heptanone oxime, (E)-3-Phenyl-2-propenal and a combination thereof.

In an embodiment of the present invention, the perfume ingredient is an aldehyde, a primary alcohol, a phenol or an oxime.

In an embodiment of the present invention, the perfume ingredient is an aldehyde selected from (2E)-2-methyl-3-phenyl-2-propenal, (E)-3-Phenyl-2-propenal and a combination thereof.

In an embodiment of the present invention, the perfume ingredient is a primary alcohol selected from (2E,6Z)-2,6-nonadien-1-ol, (E)-3,7-dimethyl-2,6-octadien-1-ol, 2-ethyl-1-hexanol, (Z)-3,7-dimethyl-2,6-octadien-1-ol, (Z)-3,7-dimethyl-2,6-octadien-1-ol, (-)-(S)-1,8-P-menthadiene-7-ol, (+-)-3-methyl-5-phenyl-1-pentanol and a combination thereof.

In an embodiment of the present invention, the perfume ingredient is a phenol selected from 5-isopropyl-2-methylphenol, 2-isopropyl-5-methylphenol, 3-propylphenol and a combination thereof.

In an embodiment of the present invention. the perfume ingredient is a oxime and the oxime is 5-methyl-3-heptanone oxime

An antimicrobial composition of the present invention may include a hydrotrope.

In aspects of the present invention, the hydrotrope is toluene sulfonate, xylene sulfonate, cumene sulfonate, diisobutyl sulfosuccinate, or a combination thereof. In further aspects, the hydrotrope is a sodium, ammonium or potassium salt of a hydrotrope selected from toluene sulfonate, xylene sulfonate, cumene sulfonate, diisobutyl sulfosuccinate, sodium salicylate, sodium acetate and sodium benzoate dipropyleneglycol n-butyl ether; or a combination thereof.

In aspects of the present invention, an antimicrobial fragrance contains at least 25% (w/v) of the perfume ingredient. That is, the perfume ingredients having a bactericidal effect represent at least 25% (w/v) of the total fragrance mixture added to the composition.

In aspects of the present invention, the composition is preferably used for reducing or eliminating microbes on external surface of human or animal body or soft and porous substrates like fabric or for hard surfaces, or for deodorant, air care, oral care and hair care applications.

The present invention encompasses a consumer product comprising antimicrobial compositions according to the present invention, wherein the consumer product is a hair care product, a body care product, a skin care product, an oral care product, a female care product, a home care product, a laundry care product, or a body cleansing product, including but not limited to shampoo, shower gel, facial cleanser, shaving gel, liquid hand soap, foaming soap, hand sanitizer, soap bar, mouthwash, toothpaste, female hygiene composition, fabric cleanser, carpet cleanser, all purpose cleanser, dishwashing detergent, fresh produce detergent, deodorant, air fresher and air disinfectant.

The present invention encompasses methods for eliminating or/and reducing the number of microbes on a surface or body part, comprising contacting the surface or body part with the antimicrobial composition of the present invention.

Further, the present invention includes use of an antimicrobial composition of the present invention for eliminating or reducing the number of microbes on a surface or body part.

In aspects of the present invention, the antimicrobial composition may be combined with a malodor neutralizing system.

The malodor neutralizing system may be (a) a composition comprising at least one ingredient selected from: (i) at least one aldehyde of formula R1CHO, wherein R1 is an aliphatic linear or branched, saturated or unsaturated carbon chain containing from 1 to 12 carbon atoms; (ii) at least one ketone of formula R2COR3, wherein R2 is an ethyl or methyl group and R3 is an aliphatic linear or branched, saturated or unsaturated carbon chain containing from 1 to 12 carbon atoms; and (iii) primary alcohols of formula R4CH2OH, wherein R4 is an aliphatic linear or branched, saturated or unsaturated carbon chain containing 1 to 12 carbon atoms, optionally substituted with an aromatic moiety; and (b) a composition comprising: (i) at least one ingredient selected from the group consisting of (2E)-1-(2,6,6-trimethyl-2-cyclohexen-1-yl)-2-buten-1-one, (2E)-1-(2,6,6-trimethyl-1-cyclohexen-1-yl)-2-buten-1-one, (2E)-1-(2,2-dimethyl-6-methylenecyclohexyl)-2-buten-1-one, (E)-1-(2,6,6-trimethyl-3-cyclohexen-1-yl)-2-buten-1-one, 1-(5,5-dimethyl-1-cyclohexen-1-yl)-4-penten-1-one, (+-)-methyl-2,2-dimethyl-6-methylene-1-cyclohexanecarboxylate, α-or β-(E)-4-(2,6,6-trimethyl-2-cyclohexen-1-yl)-3-buten-2-one (α- or β-ionone), (1E)-1-(2,6,6-trimethyl-2-cyclohexen-1-yl)-1-penten-3-one, (1E)-1-(2,6,6-trimethyl-1-cyclohexen-1-yl)-1-penten-3-one, (E)-3-methyl-4-(2,6,6-trimethyl-2-cyclohexen-1-yl)-3-buten-2-oneγ-methyl-ionone, 1-(2,6,6-trimethyl-1(2)-cyclohexen-1-yl)-1,6-heptadien-3-one and 1-(4,6,6-trimethyl-1,3-cyclohexadien-1-yl)-2-buten-1-one; and (ii) at least one nitrile ingredient selected from the group consisting of 3-phenyl-2-propenenitrile, (E/Z)-3-methyl-5-phenyl-2-pentenenitrilecitronitrile, 3,7-dimethyl-6-octenenitrilecitronellyl nitrile, 2-propyl-1-heptanenitrile, dodecanenitrile, and the mixture of 3-(2,3-dimethyl-2(3)-cyclopenten-1-yl)butanenitrile and 3-(2-methyl-3-methylene-1-cyclopentyl)butanenitrile; and c) a combination of a) and b).

An antimicrobial composition of the present invention may be combined with a malodor antagonist system. In aspects of the present invention, the antimicrobial composition may be combined with at least one compound that inhibits the activity of at least one olfactory receptor selected from the group consisting of: a DMTS olfactory receptor, an indole/skatole olfactory receptor, a butyric acid olfactory receptor, and a p-cresol olfactory receptor. The at least one compound that inhibits the activity of at least one olfactory receptor may be benzyl acetate, (1R,2R)-1,7,7-trimethyl-bicyclo[2.2.1]hept-2-yl acetate (isobornyl acetate), undec-10-enal , undec-9-enal, Virginia cedarwood essential oil, 3,7-dimethyl-2,6-octadienal, 3,7-Dimethyl-6-octen-1-ol , 3,7-dimethyloct-6-enenitrile, coumarin, (E)-1-(2,6,6-trimethyl-3-cyclohexen-1-yl)-2-buten-1-one, (E)-3-methyl-4-(2,6,6-trimethyl-2-cyclohexen-1-yl)-3-buten-2-onemethyl ionone gamma, (Z)-3,4,5,6,6-pentamethylhept-3-en-2-one, 2,6-dimethylhept-5-enal, menthone, 1-(5,5-dimethyl-1-cyclohexenyl)pent-4-en-1-one, patchouli essential oil, 2,6-nonadienal, (2-tert-butylcyclohexyl) acetate, 2-methyl-3-hexanone oxime (vertoxime), or 2-Methoxynaphthalene.

### DETAILED DESCRIPTION

According to the present invention, methods and antimicrobial compositions are provided that include a perfume ingredient having a low Log P less than or equal to 3.5, and a bactericidal effect of 5.5 log reduction in ethanol solution at a concentration of less than or equal to 0.5% in ethanol concentration; and preferably, a perfume ingredient may have a EACN less than or equal to -10.

A “perfume ingredient” as used herein, means a compound able to impart or modify, in a pleasant or positive way, the odor of a composition. In general terms, a perfume ingredient may belong to chemical classes as varied as alcohols, lactones, aldehydes, ketones, esters, ethers, acetates, nitriles, terpenoids, nitrogenous or sulphurous heterocyclic compounds and essential oils.

Another object of the invention is a perfuming composition comprising at least one ingredient selected from the group consisting of perfuming co-ingredients, a perfumery carrier and mixtures thereof, and optionally at least one perfumery adjuvant.

As liquid perfumery carrier one may cite, as non-limiting examples, a solubilizer or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery co-ingredient, other suitable perfumery carriers than those previously specified, can be also ethanol, limonene or other terpenes, isoparaffins such as those known under the trademark Isopar® (origin: Exxon Chemical) or glycol ethers and glycol ether esters such as those known under the trademark Dowanol® (origin: Dow Chemical Company). By “perfumery co-ingredient” it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to impart or modify in a positive or pleasant way the odor of a composition, and not just as having an odor.

Non-limiting examples include:
Aldehydic ingredients: decanal, dodecanal, 2-methyl-undecanal, 10-undecenal, octanal and/or nonenal;
Aromatic-herbal ingredients: eucalyptus oil, camphor, eucalyptol, menthol and/or alpha-pinene;
Balsamic ingredients: coumarine, ethylvanillin and/or vanillin;
Citrus ingredients: dihydromyrcenol, 3,7-dimethylocta-2,6-dienal, orange oil, linalyl acetate, (-)-(R)-3,7-dimethyl-6-octenenitrile, orange terpenes, limonene, 1-P-menthen-8-yl acetate and/or 1,4(8)-P-menthadiene;
Floral ingredients: Methyl dihydrojasmonate, linalool, Citronellol, phenylethanol, 3-(4-tert-butylphenyl)-2-methylpropanal, hexylcinnamic aldehyde, benzyl acetate, benzyl salicylate, tetrahydro-2-isobutyl-4-methyl-4(2H)-pyranol, beta ionone, methyl 2-(methylamino)benzoate, (E)-3-methyl-4-(2,6,6-trimethyl-2-cyclohexen-1-yl)-3-buten-2-one, hexyl salicylate, 3,7-dimethyl-1,6-nonadien-3-ol, 3-(4-isopropylphenyl)-2-methylpropanal, verdyl acetate, geraniol, P-menth-1-en-8-ol, 4-(1,1-dimethylethyl)-1-cyclohexyle acetate, 1,1-dimethyl-2-phenylethyl acetate, 4-cyclohexyl-2-methyl-2-butanol, amyl salicylate , high cis methyl dihydrojasmonate, 3-methyl-5-phenyl-1-pentanol, verdyl proprionate, geranyl acetate, tetrahydro linalool, cis-7-P-menthanol, Propyl (S)-2-(1,1-dimethylpropoxy)propanoate, 2-methoxynaphthalene, 2,2,2-trichloro-1-phenylethyl acetate, 4/3-(4-hydroxy-4-methylpentyl)-3-cyclohexene-1-carbaldehyde, amylcinnamic aldehyde, 4-phenyl-2-butanone, isononyle acetate, 4-(1,1-diméthyléthyl)-1-cyclohexyl acetate, verdyl isobutyrate and/or mixture of methylionones isomers;
Fruity ingredients: gamma undecalactone, 4-decanolide, ethyl 2-methyl-pentanoate, hexyl acetate, ethyl 2-methylbutanoate, gamma nonalactone, allyl heptanoate, 2-phenoxyethyl isobutyrate, ethyl 2-methyl-1,3-dioxolane-2-acetate and/or diethyl 1,4-cyclohexane dicarboxylate;
Green ingredients: 2,4-Dimethyl-3-cyclohexene-1-carbaldehyde, 2-tert-butyl-1-cyclohexyl acetate, (+-)-1-phenylethyl acetate, allyl (2-methylbutoxy)acetate, 4-methyl-3-decen-5-ol, diphenyl ether, (Z)-3-hexen-1-ol and/or 1-(5,5-dimethyl-1-cyclohexen-1-yl)-4-penten-1-one;
Musk ingredients: 1,4-dioxa-5,17-cycloheptadecanedione, pentadecenolide, 3-Methyl-5-cyclopentadecen-1-one, 1,3,4,6,7,8-hexahydro-4,6,6,7,8,8-hexamethyl-cyclopenta-g-2-benzopyrane, (1S,1ʹR)-2-[1-(3ʹ,3ʹ-dimethyl-1ʹ-cyclohexyl)ethoxy]-2-methylpropyl propanoate, pentadecanolide and/or (1S,1ʹR)-[1-(3ʹ,3ʹ-Dimethyl-1ʹ-cyclohexyl)ethoxycarbonyl]methyl propanoate;
Woody ingredients: 1-(octahydro-2,3,8,8-tetramethyl-2-naphtalenyl)-1-ethanone, patchouli oil, terpenes fractions of patchouli oil, (1'R,E)-2-ethyl-4-(2ʹ,2ʹ,3ʹ-trimethyl-3ʹ-cyclopenten-1ʹ-yl)-2-buten-1-ol, 2-ethyl-4-(2,2,3-trimethyl-3-cyclopenten-1-yl)-2-buten-1-ol, Methyl cedryl ketone, 5-(2,2,3-trimethyl-3-cyclopentenyl)-3-methylpentan-2-ol, 1-(2,3,8,8-tetramethyl-1,2,3,4,6,7,8,8a-octahydronaphthalen-2-yl)ethan-1-one and/or isobornyl acetate;
Other ingredients (e.g. amber, powdery spicy or watery): dodecahydro-3a,6,6,9a-tetramethyl-naphtho[2,1-b]furan and any of its stereoisomers, heliotropin, anisic aldehyde, eugenol, cinnamic aldehyde, clove oil, 3-(1,3-benzodioxol-5-yl)-2-methylpropanal and/or 3-(3-isopropyl-1-phenyl)butanal.

Perfume ingredients are not limited to those above. Perfume ingredients may also be found in reference texts such as the book by S. Arctander, Perfume and Flavor Chemicals, 1969, Montclair, New Jersey, USA, or its more recent versions, or in other works of a similar nature, as well as in the patent literature in the field of perfumery.

Non-limiting examples of a perfume ingredient include 2-ethyl-1-hexanol, (E)-3-Phenyl-2-propenal, (+-)-(3Z)-5-methyl-3-heptanone oxime or (+-)-(3E)-5-methyl-3-heptanone oxime, 5-isopropyl-2-methylphenol, 3-propylphenol, thymol, (e)-3,7-dimethyl-2,6-octadien-1-ol, (+-)-3-methyl-5-phenyl-1-pentanol, (z)-3,7-dimethyl-2,6-octadien-1-ol, (-)-(s)-1,8-p-menthadien-7-ol, (2e,6z)-2,6-nonadien-1-ol, (-)-(2E)-2-ethyl-4-[(1R)-2,2,3-trimethyl-3-cyclopenten-1-yl]-2-buten-1-ol, 2-furanmethanethiol, and (Z)-3-hexenyl formate.

Perfume ingredients according to the present invention may include 1-phenylethyl acetate, (2E)-2-methyl-3-phenyl-2-propenal, (2E,6Z)-2,6-nonadien-1-ol, 5-isopropyl-2-methylphenol, ethyl (E)-3-phenyl-2-propenoate, 3,7-dimethyl-2,6-octadienal, 3-[4-methyl-3-cyclohexen-1-yl]-1-butanol, 4-decanolide, 2-methoxy-4-propylphenol, (E)-3,7-dimethyl-2,6-octadien-1-ol, 2,4,6-trimethyl-3-cyclohexene-1-methanol, (2E)-2-methyl-3-(4-methylphenyl)-2-propen-1-ol, 2,5-dimethyl-2-indanmethanol, 2,2-dimethyl-3-[3-methyl-2,4-pentadien-1-yl]oxirane, 2-ethyl-1-hexanol, (Z)-3,7-dimethyl-2,6-octadien-1-ol, (Z)-6-nonen-1-ol, 1,8-p-menthadien-7-ol, 3-methyl-5-phenyl-1-pentanol, 2,6,6-trimethyl-1,3-cyclohexadiene-1-carbaldehyde, 2-isopropyl-5-methylphenol, 3-propylphenol, ethyl 2-hydroxybenzoate, 4-isopropyl-1-benzenemethanol, 7-propyl-2H,4H-1,5-benzodioxepin-3-one, 2-methoxy-4-(2-propen-1-yl)phenol, 3-(1,3-benzodioxol-5-yl)-2-methylpropanal, indole, 2-methoxy-4-[(1E)-1-propen-1-yl]phenol, 3-methyl-2-[(2Z)-2-penten-1-yl]-2-cyclopenten-1-one, (E)-2-hexenyl acetate, 4-(2-methyl-2-propanyl)cyclohexanone, ethyl phenylacetate, 5-methyl-3-heptanone oxime, 2-(4-methylcyclohex-3-enyl)propan-2-ol, (E)-3-phenyl-2-propenenitrile, 2-phenyl-1-propanol, (E)-3-phenyl-2-propenal, methyl 2-aminobenzoate, 4-nonanolide, 6-pentyltetrahydro-2H-pyran-2-one, 2-methyl-4-phenyl-2-butanol, 2-methyl-1-phenyl-2-propanol, 2-phenylethyl formate, (E)-2-hexen-1-ol, 4-methylphenol, 4,4a,6,7,8,8a-hexahydro-1,4-methanonaphthalen-5(1H)-one, 1-oxo-1-(2-propanyloxy)-2-propanyl 2,2-dimethylpropanoate, 2,4,6-trimethyl-3-cyclohexene-1-carbaldehyde, 3,5,6-trimethyl-3-cyclohexene-1-carbaldehyde, 3-(4-hydroxy-4-methylpentyl)-3-cyclohexene-1-carbaldehyde, 4-(4-hydroxy-4-methylpentyl)-3-cyclohexene-1-carbaldehyde, (2E)-3-phenyl-2-propen-1-ol, 2-furanmethanethiol, (Z)-3-hexenyl formate, 2-phenylethyl acetate, 1-octanol, 1,3,3-trimethylbicyclo[2.2.1]heptan-2-ol, (Z)-2-nonenal, methyl 4-methoxybenzoate, 1,3-nonanediyl diacetate, tetrahydro-3-pentyl-4(2H)-pyranyl acetate, 1-(5-propyl-1,3-benzodioxol-2-yl)ethenone, ethyl benzoate, 1,7,7-trimethylbicyclo[2.2.1]heptan-2-ol, benzyl butanoate, 1-butoxycarbonylethyl butanoate, ethyl hexanoate, 4,8-cyclododecadien-1-one, methyl (2E)-2-methyl-2-hexenoate, methyl (E)-3-phenyl-2-propenoate, (Z)-3-hexenyl acetate, 3,5,5-trimethyl-1-hexanol, methyl cyclopentylideneacetate, (Z)-4-decenal, 2,6-dimethyl-7-octen-4-one, 2,6-dimethyl-4-heptanol, 3-(2,2-dimethylpropyl)pyridine, (Z)-7-decen-4-olide, clove oil, isobutyl isobutyrate, 2,4-dimethyl-4,4a,5,9b-tetrahydroindeno[1,2-d][1,3]dioxine, 1,2-dimethoxy-4-[1-propen-1-yl]benzene, 1-(3-methyl-1-benzofuran-2-yl)ethenone, (2E,6Z)-2,6-nonadienal, 3-methylbutyl propionate, 2-methylbutyl propionate, 2-isobutyl-3-methoxypyrazine, 2-isobutyl-6-methoxypyrazine, methyl 2-hydroxybenzoate, 3-methylindole, 1-methoxy-3-hexanethiol, 8-mercapto-3-p-menthanone, 1,3,3-trimethylbicyclo[2.2.1]heptan-2-one, 1-isopropyl-4-methylbicyclo[3.1.0]hexan-3-one, 3-phenylbutanal, 6-hexyltetrahydro-2H-pyran-2-one, 7-isopropyl-2H,4H-1,5-benzodioxepin-3-one, spearmint oil or a combination thereof.

A perfume ingredient may be at least 0.001% (w/v) of the antimicrobial composition. A perfume ingredient of the present invention may be from about 0.001% to about 5.0% w/v of the antimicrobial composition. Preferably, the perfume ingredient is from about 0.01% to about 5.0% w/v of the antimicrobial composition. Further preferably, the perfume ingredient is from about 0.05% to about 5.0% w/v of the antimicrobial composition. In further aspect, the perfume ingredient is from about 0.1% to about 5.0% w/v of the antimicrobial composition.

The concentration of a perfume ingredient for a bactericidal effect of 5.5 log reduction of the present invention may be less than or equal to 0.5 % in the ethanol solution. Preferably, the concentration of a perfume ingredient for a bactericidal effect of 5.5 log reduction of the present invention may be less than or equal to 0.2% in the ethanol solution, further preferably the concentration of a perfume ingredient for a bactericidal effect of 5.5 log reduction of the present invention may be less than or equal to 0.1% in the ethanol solution.

The EACN for a perfume ingredient of the present invention may be less than or equal to -10; preferably the EACN for a perfume ingredient of the present invention may be less than or equal to -20.

The Log P of a perfume ingredient of the present invention may be less than or equal to 3.5, preferably the Log P of a perfume ingredient of the present invention may be less than or equal to 3.

A “hard surface” as used herein refers to any hard surface. Surfaces to be cleaned include kitchens and bathrooms, e.g., floors, walls, tiles, windows, cupboards, sinks, showers, shower plastified curtains, wash basins, WCs, fixtures and fittings and the like made of different materials like ceramic, vinyl, no-wax vinyl, linoleum, melamine, glass, steel, kitchen work surfaces, any plastics, plastified wood, metal or any painted or varnished or sealed surface and the like. Household hard surfaces also include household appliances including, but not limited to refrigerators, freezers, washing machines, automatic dryers, ovens, microwave ovens, dishwashers and so on. Such hard surfaces may be found both in private households as well as in commercial, institutional and industrial environments.

A “body part” as used herein refers to any part of a mammalian body that is exposed to the external environment and includes skin and mucosal surfaces. Thus, for example, a body part includes skin, oral mucosa, and teeth. In a preferred embodiment, the body part is a human body part.

A surfactant according to the present invention may be, but is not limited to, selected from the group of anionic, amphoteric, non-ionic or cationic surfactants.

Non limiting examples of anionic surfactants include sodium, potassium, or ammonium salts of alkyl sulfonates, fatty acid methylester sulfonates, alkyl benzene sulfonates, secondary alkane sulfonate, alpha olefin sulfonates, alcohol sulfates, alcohol ether sulfates, alcohol ether phosphates, sulfated alkanolamides, glyceride sulfates, fatty acids, dialkylsulfosuccinates, N-acyl-sarcosinates, N-acyl-taurates, acyl-isethionates, N-acyl-glutamates, N-acyl-glycinates, and N-acyl-alaninates.

Non limiting examples of amphoteric surfactants include alkyl betaines, alkyl amidopropyl betaines, alkyl sulfobetaines, alkyl amine oxides, lecithin (phospholipids) such as phosphatidylcholine, lysolecithin, alkyl-amphoacetate, and alkyl-amphodiacetate.

Non limiting examples of non-ionic surfactants include ethoxylated aliphatic alcohols, ethoxylated alkyl phenols, ethoxylated thiols, mixed propoxylated and ethoxylated aliphatic alcohols, ethoxylated castor oil or hydrogenated castor oil, acid ethoxylated fatty acids, fatty esters of hexitols and cyclic anhydrohexitols (e.g., sorbitan), fatty esters of ethoxylated hexitols and cyclic anhydrohexitols (e.g. polysorbate), sugar esters, alkyl polyglycosides, polyglyceryl fatty acid esters, ethoxylated amines, ethoxylated amides, and alkyl diethanolamides.

Non-ionic surfactants can be selected from the group of water-soluble triblock copolymers comprising blocks of polyethyleneglycol and polypropyleneglycol (sold under trade names such as Pluronic, Tetronic, Poloxamer, Syperonics etc.).

Surfactants can also be chosen from the group of natural biosurfactants including glycolipids (e.g., sophorolipids, mannosylerythritollipids and rhamnolipids) and saponins.

Surfactants can also be chosen from the group of cationic surfactants including alkyl quaternary ammonium salts, esterquats, linear alkyl-amines, amide-amines, ester-amines, or ethoxylated amines.

Surfactants can be used as combinations of above mentioned surfactants.

In one embodiment the anionic surfactant is sodium lauryl ether sulfate.

In another embodiment the amphoteric surfactant is cocamidopropyl betaine.

In another embodiment the non-ionic surfactant is coco-glucoside.

A surfactant may be 0.1% to 30% (w/w) of the antimicrobial composition. In certain aspects, a surfactant of the present invention may be about 0.1% to about 20% w/w of the total weight of the antimicrobial composition. In further aspect, the surfactant is about 1% to about 10% w/w of the total weight of the antimicrobial composition.

A surfactant may be 0.1% to 30% (w/w) of the antimicrobial composition. In certain aspects, the surfactant is 0.1% to 20% (w/w) of the antimicrobial composition. In further aspects, the surfactant is 0.1% to 10% (w/w) of the antimicrobial composition. In a further aspect the surfactant is 9.1% to 30% (w/w) of the antimicrobial composition. In a further aspect the surfactant is 9.1% to 20% (w/w) of the antimicrobial composition. In a further aspect the surfactant is 9.1% to 10% (w/w) of the antimicrobial composition.

An antimicrobial composition of the present invention may further comprise a hydrotrope. A hydrotrope is a substance in the presence of which the solubility of a hydrophobic compound in water is enhanced, while it does not form a microemulsion or lyotropic liquid crystals by itself.

A hydrotrope of the present invention may be about 0.5% to about 20% w/w of the total weight of the antimicrobial composition. In an aspect, the hydrotrope is about 1% to about 10% w/w of the total weight of the antimicrobial composition

In a preferred embodiment the antimicrobial composition of the present invention comprises less than 4% (w/w) hydrotrope, less than 3% (w/w) hydrotrope, less than 2% (w/w) hydrotrope, less than 1% (w/w) hydrotrope, or less.

Furthermore in another preferred embodiment, the antimicrobial composition comprises surfactant at less than or equal to 10% (w/w) of the antimicrobial composition then the hydrotrope is more than 4% (w/w) of the antimicrobial composition, preferably more than 5% (w/w) of the antimicrobial composition, preferably more than 6% (w/w) of the antimicrobial composition, preferably more than 7% (w/w) of the antimicrobial composition, preferably more than 10% (w/w) of the antimicrobial composition, preferably more than 15% (w/w) of the antimicrobial composition. Hydrotropes can be selected from the group of aryl sulfonates. In certain aspects the hydrotrope is benzene sulfonate, toluene sulfonate, xylene sulfonate, cumene sulfonate, or combinations thereof, and in the form of the corresponding sodium, ammonium or potassium salts.

The hydrotrope can also be selected from the group of diisobutyl sulfosuccinate, diisopropyl sulfosuccinate, di-n-propyl sulfosuccinate, diethyl sulfosuccinate, or combinations thereof, and in the form of the corresponding sodium, ammonium or potassium salts.

The hydrotrope can be selected from the group of benzoate, salicylate, or butyl monoglycol sulfate, and in the form of the corresponding sodium, ammonium or potassium salts.

The hydrotrope can be dipropyleneglycol-n-butyl ether.

The hydrotrope can be catechol, resorcinol, pyrogallol, hydroquinone, or 4-methoxyphenol.

The hydrotrope can be selected from the group of benzyl alcohol, urea, nicotinamide,

The hydrotrope can be sodium benzoate or sodium acetate.

The hydrotrope can be a short chain (ca. C4) alkyl polyglycoside.

The hydrotrope can be used as combinations of above mentioned hydrotropes.

Non-limiting examples of suitable hydrotropes include: toluene-sulfonate, xylenesulfonate, cumene-sulfonate, diisobutyl-sulfosuccinate, sodium salicylate, sodium acetate and sodium benzoate. An antimicrobial composition of the present invention may further comprise a solvent. According to an embodiment, the antimicrobial composition comprises a water-miscible co-solvent, preferably chosen in the group consisting of mono- and polyhydric solvents. Non limiting examples of such solvents can be found from the group containing ethanol, n-propanol, propylene glycol, hexylene glycol, dipropylene glycol, glycerol, isopropylidene glycerol, butylene glycol (1,3- butanediol), 1,2-pentanediol, 1,2-hexanediol, 1,3-propanediol, and isopropanol, and mixtures thereof. According to another embodiment the water miscible co-solvent is chosen in the group of triethylcitrate, triacetin, ethyl lactate, glycol ethers.

An antimicrobial composition of the present invention may further comprise optional ingredients such as colorants, preservatives, viscosifiers, opacifiers, emollients, humectants, antioxidants, gelling agents, gums, chelators, functional polymers, cellulose derivatives, essential oils, electrolytes, and pH adjusters.

The present invention encompasses consumer products including the antimicrobial composition such as, for example, a personal cleaning product, an oral care product, a deodorant product, a hard surface cleaning product, liquid soap, foaming soap, liquid detergent, shampoo, shower gel, facial cleanser, mouthwash, and toothpaste.

An antimicrobial composition according to the present invention may be active against Gram-negative and Gram-positive bacteria. Further, an antimicrobial composition according to the present invention may be active against the following bacteria: Escherichia coli, Salmonella sp., Pseudomonas aeruginosa, Pseudomonas fluorescens, Serratia marcescens, Klebsiella pneumoniae, Staphylococcus aureus and Listeria monocytogenes or the combination thereof.

### Malodor Antagonists

An antimicrobial composition of the present invention may be used in combination (e.g., in one composition, or in separate compositions that are administered simultaneously or near in time) with a malodor antagonist system. A malodorous compound may activate at least one olfactory receptor associated with the malodor. Without intending to be limited to any particular theory, malodors are usually complex mixtures of more than one malodorous compound which may include various amines, thiols, sulfides, short chain aliphatic and unsaturated acids, e.g. fatty acids, and their derivatives. In one aspect, the at least one olfactory receptor is an olfactory receptor disclosed in International Patent Application Publication No. WO2019/101821 A1. In an alternate aspect, the at least one olfactory receptor is an olfactory receptor disclosed in International Patent Application Publication No. WO2018/091686 A1. In an alternate aspect, the at least one olfactory receptor is an olfactory receptor disclosed in International Patent Application Publication No. WO2018/091686 A1.

In one aspect, the inhibition of the at least one olfactory receptor inhibits, reduces, suppresses, the perception of a malodor in a consumer.

As used herein, the terms “antagonists,” “inhibitor,” “blockers, ” “suppressors, ” “counteractants” and “modulators” of olfactory receptors are used interchangeably to refer to inhibitory, blocking, suppressing, or modulating molecules identified using in vivo, ex vivo and in vitro assays for olfactory transduction, e.g., ligands, antagonists, and their homologs and mimetics. Inhibitors are compounds that, e.g., bind to, partially or totally block stimulation, decrease, suppress, prevent, delay activation, inactivate, desensitize, or down regulate olfactory transduction, e.g., antagonists. Activators are compounds that, e.g., bind to, stimulate, increase, open activate, facilitate, enhance activation, sensitize, or up regulate olfactory transduction, e.g., agonists. Modulators include compounds that, e.g., alter the interaction of a receptor with: extracellular proteins that bind activators or inhibitor (e.g., odourant-binding proteins, ebnerin and other members of the hydrophobic carrier family); G proteins; kinases (e.g., homologs of rhodopsin kinase and beta adrenergic receptor kinases that are involved in deactivation and desensitization of a receptor); and arrestins, which also deactivate and desensitize receptors.

The ability of compounds and methods of the present disclosure to inhibit or antagonize the at least one olfactory receptor may be determined by any suitable method readily selected by one of ordinary skill in the art, such as, for example, via an ex vivo cultured neuron assay, or via an in vitro assay using a cell line that expresses a butyric acid olfactory receptor.

As used herein, the term “olfactory receptor”, or “OR” refers to one or more members of a family of G protein-coupled receptors (GPCRs) that are expressed in olfactory cells. Olfactory receptor cells can also be identified on the basis of morphology or by the expression of proteins specifically expressed in olfactory cells. OR family members may have the ability to act as receptors for odorants and induce an olfactory transduction cascade.

In one aspect, the at least one compound that inhibits the activity of at least one olfactory receptor is selected from the group consisting of: benzyl acetate, isobornyl acetate, undec-10-enal, undec-9-enal, Virginia cedarwood essential oil, 3,7-dimethyl-2,6-octadienal, 3,7-Dimethyl-6-octen-1-ol, 3,7-dimethyloct-6-enenitrile, coumarin, (2E)-1-(2,2-dimethyl-6-methylenecyclohexyl)-2-buten-1-one, methyl ionone gamma, (Z)-3,4,5,6,6-pentamethylhept-3-en-2-one, 2,6-dimethylhept-5-enal, menthone, 1-(5,5-dimethyl-1-cyclohexenyl)pent-4-en-1-one, patchouli essential oil, 2,6-nonadienal, (2-tert-butylcyclohexyl) acetate, vertoxime, and 2-Methoxynaphthalene.

Examples of other compounds capable of inhibiting the activity of at least one olfactory receptor selected from the group consisting of: a DMTS olfactory receptor, an indole/skatole olfactory receptor, a butyric acid olfactory receptor, and a p-cresol olfactory receptor include the compounds disclosed in International Patent Application Publication No. WO2019/101821 A1.

Additional examples of other compounds capable of inhibiting the activity of at least one olfactory receptor selected from the group consisting of: a DMTS olfactory receptor, an indole/skatole olfactory receptor, a butyric acid olfactory receptor, and a p-cresol olfactory receptor include the compounds disclosed in International Patent Application Publication No. WO2018/091686 A1.

In one aspect, an at least one compound capable of inhibiting the activity of a DMTS olfactory receptor may be selected from the compounds capable of inhibiting the activity of a DMTS olfactory receptor disclosed in International Patent Application Publication No. WO2019/101821 A1.

In one aspect, an at least one compound capable of inhibiting the activity of a butyric acid olfactory receptor may be selected from the compounds capable of inhibiting the activity of a butyric acid olfactory receptor disclosed in International Patent Application Publication No. WO2019/101821 A1.

In one aspect, an at least one compound capable of inhibiting the activity of an indole/ skatole olfactory receptor may be selected from the compounds capable of inhibiting the activity of an indole/ skatole olfactory receptor disclosed in International Patent Application Publication No. WO2019/101821 A1.

In one aspect, an at least one compound capable of inhibiting the activity of a p-cresol olfactory receptor may be selected from the compounds capable of inhibiting the activity of a p-cresol olfactory receptor disclosed in International Patent Application Publication No. WO2018/091686 A1.

In one aspect, the malodor antagonist system is present in the antimicrobial composition in an amount from 30 to 50% wt%, relative to the antimicrobial composition.

In one aspect, the malodor antagonist system is present in the antimicrobial composition in an amount from 30 to 45, or alternatively, in an amount from 30 to 40, or alternatively, in an amount from 30 to 35 wt%, relative to the antimicrobial composition.

In one aspect, the malodor antagonist system is present in the antimicrobial composition in an amount from 35 to 50, or alternatively, from 40 to 50, or alternatively, from 45 to 50 wt%, relative to the antimicrobial composition.

In one aspect, the malodor antagonist system is present in the antimicrobial composition at 30, or 35, or 40, or 45, or 50 wt%, relative to the antimicrobial composition.

### Malodor Neutralizing System

An antimicrobial composition of the present invention may be used in combination (e.g., in one composition, or in separate compositions that are administered simultaneously or near in time) with a malodor neutralizing system. A malodor neutralizing system limits, decreases or eliminates the perception of a malodor by reacting with various chemical compounds that may be responsible for the malodor. The reactions result in reduction of the malodor material’s airborne levels and consequent reduction in the perception of the malodor.

In one aspect, the at least one malodor neutralizing system is selected from the group consisting of: a) a composition comprising at least one ingredient selected from the group consisting of: (i) at least one aldehyde of formula R¹CHO, wherein R¹ is an aliphatic linear or branched, saturated or unsaturated carbon chain containing from 1 to 12 carbon atoms; (ii) at least one ketone of formula R²COR³, wherein R² is an ethyl or methyl group and R³ is an aliphatic linear or branched, saturated or unsaturated carbon chain containing from 1 to 12 carbon atoms; and (iii) primary alcohols of formula R⁴CH₂OH, wherein R⁴ is an aliphatic linear or branched, saturated or unsaturated carbon chain containing 1 to 12 carbon atoms, optionally substituted with an aromatic moiety; b) a composition comprising: (i) at least one ingredient selected from the group consisting of (2E)-1-(2,6,6-trimethyl-2-cyclohexen-1-yl)-2-buten-1-oneα, (2E)-1-(2,6,6-trimethyl-1-cyclohexen-1-yl)-2-buten-1-one, (2E)-1-(2,2-dimethyl-6-methylenecyclohexyl)-2-buten-1-oneγ, (E)-1-(2,6,6-trimethyl-3-cyclohexen-1-yl)-2-buten-1-oneδ, 1-(5,5-dimethyl-1-cyclohexen-1-yl)-4-penten-1-one, (+-)-methyl-2,2-dimethyl-6-methylene-1-cyclohexanecarboxylate, α- or β-(E)-4-(2,6,6-trimethyl-2-cyclohexen-1-yl)-3-buten-2-one (α- or β-ionone), (1E)-1-(2,6,6-trimethyl-2-cyclohexen-1-yl)-1-penten-3-one, (1E)-1-(2,6,6-trimethyl-1-cyclohexen-1-yl)-1-penten-3-one, (E)-3-methyl-4-(2,6,6-trimethyl-2-cyclohexen-1-yl)-3-buten-2-one, 1-(2,6,6-trimethyl-1(2)-cyclohexen-1-yl)-1,6-heptadien-3-one and 1-(4,6,6-trimethyl-1,3-cyclohexadien-1-yl)-2-buten-1-one; and (ii) at least one nitrile ingredient selected from the group consisting of 3-phenyl-2-propenenitrile, (E/Z)-3-methyl-5-phenyl-2-pentenenitrile, 3,7-dimethyl-6-octenenitrile, 2-propyl-1-heptanenitrile, dodecanenitrile, and the mixture of 3-(2,3-dimethyl-2(3)-cyclopenten-1-yl)butanenitrile and 3-(2-methyl-3-methylene-1-cyclopentyl)butanenitrile; and c) a combination of a) and b).

Examples of compositions comprising at least one ingredient selected from the group consisting of: (i) at least one aldehyde of formula R¹CHO, wherein R¹ is an aliphatic linear or branched, saturated or unsaturated carbon chain containing from 1 to 12 carbon atoms; (ii) at least one ketone of formula R²COR³, wherein R² is an ethyl or methyl group and R³ is an aliphatic linear or branched, saturated or unsaturated carbon chain containing from 1 to 12 carbon atoms; and (iii) primary alcohols of formula R⁴CH₂OH, wherein R⁴ is an aliphatic linear or branched, saturated or unsaturated carbon chain containing 1 to 12 carbon atoms, optionally substituted with an aromatic moiety may be found in U.S. Pat. No. 8,772,354.

Examples of compositions comprising at least one ingredient selected from the group consisting of (i) at least one ingredient selected from the group consisting of (2E)-1-(2,6,6-trimethyl-2-cyclohexen-1-yl)-2-buten-1-one, (2E)-1-(2,6,6-trimethyl-1-cyclohexen-1-yl)-2-buten-1-one, (2E)-1-(2,2-dimethyl-6-methylenecyclohexyl)-2-buten-1-one, (E)-1-(2,6,6-trimethyl-3-cyclohexen-1-yl)-2-buten-1-one, 1-(5,5-dimethyl-1-cyclohexen-1-yl)-4-penten-1-one, (+-)-methyl-2,2-dimethyl-6-methylene-1-cyclohexanecarboxylate, α- or β-(E)-4-(2,6,6-trimethyl-2-cyclohexen-1-yl)-3-buten-2-one (α- or β-ionone), (1E)-1-(2,6,6-trimethyl-2-cyclohexen-1-yl)-1-penten-3-one, (1E)-1-(2,6,6-trimethyl-1-cyclohexen-1-yl)-1-penten-3-one, (E)-3-methyl-4-(2,6,6-trimethyl-2-cyclohexen-1-yl)-3-buten-2-one, 1-(2,6,6-trimethyl-1(2)-cyclohexen-1-yl)-1,6-heptadien-3-one and 1-(4,6,6-trimethyl-1,3-cyclohexadien-1-yl)-2-buten-1-one; and (ii) at least one nitrile ingredient selected from the group consisting of 3-phenyl-2-propenenitrile, citronitrile, citronellyl nitrile, 2-propyl-1-heptanenitrile, dodecanenitrile, and the mixture of 3-(2,3-dimethyl-2(3)-cyclopenten-1-yl)butanenitrile and 3-(2-methyl-3-methylene-1-cyclopentyl)butanenitrile may be found in U.S. Pat. Application Publication No. 2017/0266334 A1.

In one aspect, the at least one malodor neutralizing system is present in the antimicrobial composition in an amount from 5 to 20% wt%, relative to the antimicrobial composition.

In one aspect, the at least one malodor neutralizing system is present in the antimicrobial composition in an amount from 5 to 19, or alternatively, in an amount from 5 to 18, or alternatively, in an amount from 5 to 17, or alternatively, in an amount from 5 to 16, or alternatively, in an amount from 5 to 15, or alternatively, in an amount from 5 to 14, or alternatively, in an amount from 5 to 13, or alternatively, in an amount from 5 to 12, or alternatively, in an amount from 5 to 11, or alternatively, in an amount from 5 to 10, or alternatively, in an amount from 5 to 9, or alternatively, in an amount from 5 to 8, or alternatively, in an amount from 5 to 7, or alternatively, in an amount from 5 to 6 wt%, relative to the antimicrobial composition.

In one aspect, the at least one malodor neutralizing system is present in the antimicrobial composition in an amount from 6 to 20, or alternatively, in an amount from 7 to 20, or alternatively, in an amount from 8 to 20, or alternatively, in an amount from 9 to 20, or alternatively, in an amount from 10 to 20, or alternatively, in an amount from 11 to 20, or alternatively, in an amount from 12 to 20, or alternatively, in an amount from 13 to 20, or alternatively, in an amount from 14 to 20, or alternatively, in an amount from 15 to 20, or alternatively, in an amount from 16 to 20, or alternatively, in an amount from 17 to 20, or alternatively, in an amount from 18 to 20, or alternatively, in an amount from 19 to 20 wt%, relative to the antimicrobial composition.

In one aspect, the at least one malodor neutralizing system is present in the malodor counteracting composition at 5, or 6, or 7, or 8, or 9, or 10, or 11, or 12, or 13, or 14, or 15, or 16, or 17, or 18, or 19, or 20 wt%, relative to the antimicrobial composition.

The present invention is illustrated by, but is not limited to, the following examples.

### EXAMPLES

### Example 1: Determination of Antimicrobial Efficacy of Fragrance Materials In Ethanol Solution

### a. Preparation of Bacterial Suspensions

Bacterial suspensions of E. coli ATCC 10536 were prepared for antimicrobial testing as follows. Stock cultures stored at -80° C. were sub-cultured onto Tryptic Soy Agar (TSA) plate, and incubated at 37° C. for 24 h to obtain single colonies. Single colonies of the primary cultures were streaked onto TSA plates and incubated at 37° C. for 24 h to prepare the secondary cultures. Single colonies of secondary cultures were inoculated into 50 mL of Tryptic Soy broth (TSB), incubated at 37° C. 180 rpm for 18 h. Aliquots (0.5 ml) of the 18 h culture were inoculated into 50 ml of fresh TSB, and incubated at 37° C. 180 rpm for 2- 3 hours. When the OD600 nm value of the broth reached 1-2, cells were harvested by centrifugation at 5,000 rpm for 10 min, and then resuspended in the same fresh broth media to achieve the target level of 1-5 × 10⁸ colony forming unit (CFU)/mL. This suspension was used for further antimicrobial tests.

### b. Determination of the Bactericidal Activity of Perfume Ingredients In Ethanol Solutions

Unless indicated otherwise, log reduction testing according to the present invention is determined in accordance with the following method.

Bacterial contact time (BCT) test based on European standard EN-1276 was used to determine dose dependent bactericidal activity of perfume ingredients in 20% ethanol solutions.

Perfume ingredients of various concentrations (doses) were prepared in 40% ethanol solutions. Eleven replicates of each sample were added into 96-well microtiter plates (120 µl per well) in column 1-11 of one row, with row B as the control sample of the ethanol solution. Then, 120 µl of cell suspension at a concentration of approximately 1-5 × 10⁸ CFU/ML (as prepared above) were added to each well of the microtiter plate. A specified contact time (45 s) was allowed for target bacterial strains. At the end of the contact time, serial dilutions were prepared in 96 well plates with growth media: 3 times of 1 in 10 dilutions and followed by 17 times of 1 in 2 dilutions. Each plate was sealed and incubated at 37° C. under agitation (180 rpm). After incubation, turbidity of the wells (OD 600 nm) was recorded by Tecan microplate reader. Turbid cells were regarded as positive growth of viable cells. The total number of viable cells (log CFU/mL) for each sample was calculated. And the log reduction for each test composition at the final concentration (dose) was calculated against the control sample of the 20% ethanol solution.

### Example 2: Measurement of Equivalent Alkane Carbon Number (EACN) of a Perfume Ingredient In Model System

EACN values provide information on polarity and surface activity of apolar ingredients. Tchakalova and Fieber (J. of Surfactants and Detergents, 2012, 15(2):167-177) classified perfume ingredients based on values of EACNₘᵢₓ, the EACN of a mixture consisting of a test perfume ingredient and a reference oil (isopropyl myristate). The same model system and method were used to measure the EACN values of perfume ingredients with antimicrobial activities. Different perfume ingredients were added to the model microemulsion system shown in Table 1, in which the nonionic pentaethylene glycol mono n-decyl ether (C10E5) was used as surfactant. Phase transition temperatures of the system from Winsor I to Winsor III, and from Winsor III to Winsor II were determined using a water bath. Alkanes with different alkane carbon numbers (octane, decane, dodecane and hexadecane) were used to establish a linear correlation curve between EACN and the PIT, which is the average of the transitions from Winsor I to Winsor III, and from Winsor III to Winsor II, respectively. EACNₘᵢₓ was then calculated based on the linear relationship and the measured PIT for systems containing different perfume ingredients. The EACN of the pure perfume ingredient was calculated based on the molar ratio of the tested PRMs in the oil phase using following equation:

$$E A C {N}_{m i x} = E A C N \times n + E A C {N}_{r e f} \times {n}_{r e f}$$

wherein n and n_{ref} are the molar fractions of tested perfume ingredient and the reference ingredient (i.e., isopropyl myristate) in the oil phase, respectively.

TABLE 1

Compositions for EACN measurement

| Model system | Conc. (% wt) |
|---|---|
| C10E5 | 10 |
| H₂O | 45 |
| Isopropyl Myristate | 42 |
| PERFUME INGREDIENT | 3 |

### Example 3: Determination of the Log P of Perfume Ingredients

The partition coefficient (P) is defined as the ratio of the concentrations (C) to the equilibrium of a dissolved and highly diluted substance in a biphasic system consisting of two almost immiscible solvents.

In the case of n-octanol and water:

$$P = C ( n - o c t a n o l ) / C ( w a t e r )$$

The partition coefficient P is the quotient of two concentrations, it is generally indicated in its logarithmic form, base 10 (LogP).

The log P values of different perfume ingredients were measured using standard high performance liquid chromatography method, according to the reference (OECD, guideline No. 117, adopted on 13 Apr. 2004, Partition coefficient (n-octanol-water), High Performance Liquid Chromatography (HPLC) Method).

### Example 4: Determination of the Antimicrobial Efficacy of Perfume Ingredients In different surfactant bases

### a. Preparation of Test Samples

The surfactant bases tested included 1) sodium lauryl ether sulfate (SLES) and cocamidopropyl betaine (CAPB) at 3:1 mass ratios; 2) SLES, CAPB and coco-glucoside at 2:1:1 mass ratios. Test samples were prepared by mixing perfume ingredients with the surfactant bases and vigorously stirred for 24 hrs. Concentration of perfume ingredients in the bases was 0.5% wt. Samples with transparent appearance were selected for antimicrobial testing.

### b. Measurement of Antimicrobial Efficacy of Fragrance Materials In Surfactant Systems

Antimicrobial efficacy was tested against a representative Gram-negative bacterial strain, Escherichia coli ATCC 10536, using robotic bacteria contact time (BCT) test based on European standard EN-1276 and EP2787827(B1).

Preparation of screening plate and dilution plate: Aliquots (270 µl) of compositions were dispensed into the wells of a 96-well microtiter plate (MTP) along two columns (B1-H1, and B7-H7), and 270 µl of MilliQ water was added to well A1 and A7 as the control samples. This MTP was labelled as the “Screening plate”. In another MTP, labelled as the “Dilution plate”, 270 µL of Dey-Engley (D/E) neutralizing solution was added to column 1 and column 7. 270 µl of tryptone diluent solution was added to columns 2-6 and columns 8-12 of the Dilution MTP by a Hamilton robotic liquid handling station.

BCT test & neutralization & dilution: Bacterial stock (30 µl) was then added to columns 1 of the ‘Screening plate’ and mixed by a Hamilton robotic liquid handling station. After a contact time of 45 seconds, 30 µl of the mixtures in column 1 were transferred into the corresponding wells of column 1 of the ‘Dilution plate’. After neutralizing for 5 minutes in the D/E neutralizing solution, 30 µl of the neutralized mixtures were transferred from column 1 to column 2 of the Dilution MTP and mixed, followed by transferring 30 µl of the mixtures from column 2 into column 3. This process was repeated serially diluting the bacteria suspensions across the plate to column 6. Bacterial stock (30 µl) was then added to columns 7 of the ‘Screening plate’ and mixed by a Hamilton robotic liquid handling station. After a contact time of 45 seconds, 30 µl of the mixtures in column 7 were transferred into the corresponding wells of column 7 of the ‘Dilution plate’. After neutralizing for 5 minutes in the D/E neutralizing solution, 30 µl of the mixtures were transferred from column 7 to column 8 of the Dilution MTP and mixed, followed by transferring further 30 µl mixtures from column 8 into column 9. This process was repeated serially diluting the bacteria suspensions across the plate to column 12.

Plating: 30 µl volumes from each well in the Dilution MTP were transferred onto four Tryptone Soya Agar (TSA) plates. The TSA plates were allowed to stand for about two hours so that the 30 µl inocula spots could dry and the plates were then inverted and incubated overnight at 37° C. for 24 hours. After incubation, colonies were counted.

Calculating log reduction: Select the dilutions with colony counts, and calculate the viable cell counts (CFU/mL) of mixture in ‘Screening plate’, and log reduction against the control MilliQ sample was calculated.

### Results

Based on the antimicrobial test results in ethanol solutions, perfume ingredients were divided into two groups:

A. Perfume ingredients with high bactericidal activity in ethanol solutions: at least a 5.5 log reduction in bacterial viability at less than or equal to 0.1% (w/v) of the bactericidal perfume ingredients in ethanol solution against at least one Gram-negative bacterium, preferably at least one of Escherichia coli, Salmonella sp., Pseudomonas aeruginosa, Pseudomonas fluorescens, Serratia marcescens, and Klebsiella pneumoniae, when tested according to the test procedure as described in Example 1.

B. Perfume ingredients with medium bactericidal activity in ethanol solutions: at least a 5.5 log reduction in bacterial viability at a concentration within 0.1 - 0.5% (w/v) of the bactericidal perfume ingredients in ethanol solution against at least one Gram-negative bacterium, preferably at least one of Escherichia coli, Salmonella sp., Pseudomonas aeruginosa, Pseudomonas fluorescens, Serratia marcescens, and Klebsiella pneumoniae, when tested according to the test procedure as described in Example 1.

TABLE 2

Perfume ingredient with high bactericidal activity

| Perfume ingredient | EACN | Log P |
|---|---|---|
| (+-)-1-phenylethyl acetate | -0.99 | 2.22 |
| undecanal | 4.59 | 4.56 |
| (2E)-2-methyl-3-phenyl-2-propenal | -12.17 | 2.56 |
| (2E,6Z)-2,6-nonadien-1-ol | -35.12 | 2.68 |
| 5-isopropyl-2-methylphenol | -43.89 | 2.52 |
| Ethyl(E)-3-phenyl-2-propenoate | -5.36 | 3 |
| (2Z)-3,7-dimethyl-2,6-octadienal OR (2E)-3,7-dimethyl-2,6-octadienal | -5.09 | 2.68 |
| (+)-(3R)-3-[(1R)-4-methyl-3-cyclohexen-1-yl]-1-butanol OR (+)-(3S)-3-[(1R)-4-methyl-3-cyclohexen-1-yl]-1-butanol | -34.57 | 3.46 |
| (-)-(2E)-2-ethyl-4-[(1R)-2,2,3-trimethyl-3-cyclopenten-1-yl]-2-buten-1-ol | -23.86 | 4.44 |
| (+-)-4-decanolide | -4.96 | 3.02 |
| 2-methoxy-4-propyl phenol | -34.16 | 2.39 |
| (E)-3,7-dimethyl-2,6-octadien-1-ol | -28.65 | 2.97 |
| (+-)-2,4,6-trimethyl-3-cyclohexene-1-methanol | -25.02 | 3.3 (predicted) |
| (2E)-2-methyl-3-(4-methylphenyl)-2-propen-1-ol | -36.15 | 2.29 |
| (+-)-2,5-dimethyl-2-indanmethanol | -33.01 | 3.12 |
| (+-)-(2,2-dimethyl-3-[(2Z)-3-methyl-2,4-pentadien-1-yl]oxirane OR (+-)-(2,2-dimethyl-3-[(2E)-3-methyl-2,4-pentadien-1-yl]oxirane | -1.45 | 2.92 |
| 2-ethyl-1-hexanol | -22.63 | 2.73 |
| (Z)-3,7-dimethyl-2,6-octadien-1-ol | -28.26 | 2.91 |
| (Z)-6-nonen-1-ol | -29.91 | 3.4 |
| (-)-(S)-1,8-P-menthadien-7-ol | -30.83 | 2.68 |
| (+-)-3-methyl-5-phenyl-1-pentanol | -37.05 | 3 |
| 2,6,6-trimethyl-1,3-cyclohexadiene-1-carbaldehyde | -6.62 | 2.57 |
| 2 /3 //4-(5,5,6-trimethylbicyclo[2.2.1]hept-2-yl)-1-cyclohexanol(A/B/C) + 2-(1,7,7-trimethylbicyclo[2.2.1]hept-2-yl)-1-cyclohexanol (D) | -16.52 | 5.23 |
| (+-)-3,7-dimethyl-3-octanol | -4.83 | 3.78 |
| 2-isopropyl-5-methylphenol | -43.68 | 2.57 |
| 3-propylphenol | <-29.09 | 2.1 |

TABLE 3

Perfume ingredient with medium bactericidal activity

| Perfume ingredient | EACN | Log P |
|---|---|---|
| 4-isopropyl-1-benzenemethanol | -36.04 | 2.23 |
| 7-propyl-2H,4H-1,5-benzodioxepin-3-one | -40.99 | 2.79 |
| 2-methoxy-4-(2-propen-1-yl)phenol | -31.24 | 2.14 |
| (+-)-3-(1,3-benzodioxol-5-yl)-2-methylpropanal | -11.56 | 1.28 |
| indole | <-24.93 | 1.74 |
| 2-methoxy-4-[(1E)-1-propen-1-yl]phenol | -42.88 | 2.15 |
| 3-methyl-2-[(2Z)-2-penten-1-yl]-2-cyclopenten-1-one | -8.51 | 2.61 |
| (E)-2-hexenyl acetate | 1.12 | 2.59 |
| 4-(2-methyl-2-propanyl)cyclohexanone | -3.78 | 2.77 |
| ethyl phenylacetate | -7.26 | 2.1 |
| (+-)-(3Z)-5-methyl-3-heptanone oxime OR (+-)-(3E)-5-methyl-3-heptanone oxime | -13.67 | 2.45 |
| (+-)-alpha-terpineol | -16.69 | 2.91 |
| (E)-3-phenyl-2-propenenitrile | -14.36 | 1.98 |
| (+-)-2-phenyl-1-propanol | -29.67 | 1.74 |
| (E)-3-Phenyl-2-propenal | -13.35 | 1.5 |
| methyl 2-aminobenzoate | -22.57 | 1.83 |
| (+-)-4-nonanolide | -11.24 | 2.45 |
| (+-)-6-pentyltetrahydro-2H-pyran-2-one | -13.27 | 2.22 |
| 2-methyl-4-phenyl-2-butanol | -24.94 | 2.69 |
| 2-methyl-1-phenyl-2-propanol | -17.89 | 1.84 |
| 2-phenylethyl formate | -6.21 | 1.9 |
| (2E)-2-hexen-1-ol | -21.91 | 1.73 |
| 4-methylphenol | <-21.95 | 1.52 |
| (1RS,2SR,7RS,8SR)-tricyclo[6.2.1.0^{~}2,7^{~}]undec-9- en-3-one OR (1RS,2RS,7S,8SR)-tricyclo[6.2.1.0^{~}2, 7^{~}] undec-9-en-3-one | -5.81 | 2.53 |
| (2E)-3-phenyl-2-propen-1-ol | < -28.19 | 1.79 |

Antimicrobial performances of perfume ingredients were tested in model base 1-4, as shown in Table 4 to Table 6.

Table 7 shows antibacterial performances of binary mixtures of perfume ingredient with low EACN and low log P values tested in model base 2.

Table 8 shows the compositions of complex mixtures of antimicrobial perfume ingredient mixtures designed based on sensory performances.

Eight model surfactant bases are designed to test antimicrobial performances of antibacterial perfume ingredients, and their mixtures. Table 9 shows the compositions of the eight model bases.

Table 10 shows the antimicrobial activities of complex mixtures in model surfactant bases 5-8.

TABLE 4

Antimicrobial efficacy of Perfume ingredient with high bactericidal activity in ethanol against E.coli ATCC10536 in sodium lauryl ether sulfate (SLES) and cocamidopropyl betaine (CAPB) base

| Perfume ingredient (0.5%) | EACN | Log P | Log reduction in Base 1 (Log CFU/mL) |
|---|---|---|---|
| 2-ethyl-1-hexanol | -22.63 | 2.73 | 3.6 |
| 5-isopropyl-2-methylphenol | -43.89 | 2.52 | 5.3 |
| 3-propylphenol | <-29.09 | 2.1 | >5.5 |
| 2-isopropyl-5-methylphenol | -43.68 | 2.57 | >5.5 |
| (E)-3,7-dimethyl-2,6-octadien-1-ol | -28.65 | 2.97 | >5.4 |
| (+-)-3-methyl-5-phenyl-1-pentanol | -37.05 | 3.0 | 5.0 ± 2.1 |
| (Z)-3,7-dimethyl-2,6-octadien-1-ol | -28.26 | 2.91 | >5.4 |
| (-)-(S)-1,8-P-menthadien-7-ol | -30.83 | 2.68 | 4.2 ± 0.1 |
| (2E,6Z)-2,6-nonadien-1-ol | -35.12 | 2.68 | 5.6 |
| Ethyl(E)-3-phenyl-2-propenoate | -5.26 | 3.0 | 1.9 ± 1.0 |
| (+-)-3,7-dimethyl-3-octanol | -4.83 | 3.78 | 1.9 ± 1.6 |
| (+-)-4-decanolide | -4.96 | 3.02 | 0.2 ± 0.3 |
| (+-)-1-phenylethyl acetate | -0.99 | 2.22 | 0.6 ± 1.0 |
| (-)-(2E)-2-ethyl-4-[(1R)-2,2,3-trimethyl-3-cyclopenten-1-yl]-2-buten-1-ol | -23.86 | 4.44 | -0.2 |
| 2/3/4-(5,5,6-trimethylbicyclo[2.2.1]hept-2-yl)-1-cyclohexanol(A/B/C) +2-(1,7,7-trimethylbicyclo[2.2.1]hept-2-yl)-1-cyclohexanol (D) | -16.52 | 5.23 | 0.4 |

TABLE 5

Antimicrobial efficacy of Perfume ingredient with medium bactericidal activity in ethanol against E. coli ATCC10536 in model Base 1

| Perfume ingredient (0.5%) | EACN | Log P | Log reduction in Base 1 (Log CFU/mL) |
|---|---|---|---|
| (+-)-(3Z)-5-methyl-3-heptanone oxime OR (+-)-(3E)-5-methyl-3-heptanone oxime | -13.67 | 2.45 | 3.3 |
| (E)-3-Phenyl-2-propenal | -13.35 | 1.5 | 5.1 |
| 2-methoxy-4-(2-propen-1-yl)phenol | -31.24 | 2.14 | 0.2 |
| 2-methoxy-4-[(1E)-1-propen-1-yl]phenol | -42.88 | 2.15 | 0 |
| 4-isopropyl-1-benzenemethanol | -36.04 | 2.23 | 1.3 |
| 7-propyl-2H,4H-1,5-benzodioxepin-3-one | -40.99 | 2.79 | 0 |
| (+-)-alpha-terpineol | -16.69 | 2.91 | 1.6 |
| indole | <-24.93 | 1.74 | 1.9 |
| (2E)-3-phenyl-2-propen-1-ol | < -28.19 | 1.79 | 0.1 |
| 4-methylphenol | <-21.95 | 1.52 | 0.1 |
| (2E)-2-hexen-1-ol | -21.91 | 1.73 | -0.2 |
| (+-)-2-phenyl-1-propanol | -29.67 | 1.74 | -0.3 |
| 2-methyl-4-phenyl-2-butanol | -24.94 | 2.69 | -0.4 |
| 2-methyl-1-phenyl-2-propanol | -17.89 | 1.84 | -0.1 |
| 2-phenylethyl formate | -6.21 | 1.9 | 0.3 |

TABLE 6

Antimicrobial efficacy of Perfume ingredient with high bactericidal activity in ethanol against E. coli ATCC10536 in model bases 2-4

| Perfume ingredient (0.5%) | EACN | Log P | Log reduction (Log CFU/mL) | | |
|---|---|---|---|---|---|
| | | | Base 2 | Base 3 | Base 4 |
| Ethyl(E)-3-phenyl-2-propenoate | -5.36 | 3 | 0 | Insoluble | Insoluble |
| (+-)-3,7-dimethyl-3-octanol | -4.83 | 3.78 | 0 | 0.3 | 0.4 |
| (+-)-4-decanolide | -4.96 | 3.02 | 0 | 0.6 | 0.25 |
| (+-)-1-phenylethyl acetate | -0.99 | 2.22 | 0 | 0.2 | Insoluble |
| (-)-(2E)-2-ethyl-4-[(1R)-2,2,3-trimethyl-3-cyclopenten-1-yl]-2-buten-1-ol | -23.86 | 4.44 | -1.0 | Insoluble | Insoluble |
| 2/3/4-(5,5,6-trimethylbicyclo[2.2.1]hept-2-yl)-1-cyclohexanol(A/B/C) +2-(1,7,7-trimethylbicyclo[2.2.1]hept-2-yl)-1-cyclohexanol (D) | -16.52 | 5.23 | -1.0 | Insoluble | -0.2 |
| (E)-3,7-dimethyl-2,6-octadien-1-ol | -28.65 | 2.97 | 0.5 | 4.2 | Insoluble |
| (+-)-3-methyl-5-phenyl-1-pentanol | -37.05 | 3 | 0 | 2.9 | 2.8 |
| (Z)-3,7-dimethyl-2,6-octadien-1-ol | -28.26 | 2.91 | 0.5 | 4.9 | 4.5 |
| (-)-(S)-1,8-P-menthadien-7-ol | -30.83 | 2.68 | 0 | 1.4 | 2.2 |
| (2E,6Z)-2,6-nonadien-1-ol | -35.12 | 2.68 | 0.5 | 3.9 | >4.5 |
| Control (Base only) | | | 0 | 0 | 0 |

TABLE 7

Antimicrobial activity of binary mixtures of perfume ingredients with low EACN and low log P values in model base 2

| Perfume Ingredient A | Perfume Ingredient B | Log reduction in Base 2 (Log CFU/mL) |
|---|---|---|
| 5-isopropyl-2-methylphenol | (E)-3,7-dimethyl-2,6-octadien-1-ol | 4.7 |
| 5-isopropyl-2-methylphenol | (+-)-3-methyl-5-phenyl-1-pentanol | 5.1 |
| 5-isopropyl-2-methylphenol | (Z)-3,7-dimethyl-2,6-octadien-1-ol | 4.6 |
| 5-isopropyl-2-methylphenol | (2E,6Z)-2,6-nonadien-1-ol | 4.8 |
| 3-propylphenol | (E)-3,7-dimethyl-2,6-octadien-1-ol | 4.6 |
| 3-propylphenol | (+-)-3-methyl-5-phenyl-1-pentanol | 4.1 |
| 3-propylphenol | (Z)-3,7-dimethyl-2,6-octadien-1-ol | 4.4 |
| 3-propylphenol | (2E,6Z)-2,6-nonadien-1-ol | 2.6 |
| 2-isopropyl-5-methylphenol | (+-)-3-methyl-5-phenyl-1-pentanol | >5.5 |
| (+-)-(3Z)-5-methyl-3-heptanone oxime OR (+-)-(3E)-5-methyl-3-heptanone oxime | 5-isopropyl-2-methylphenol | >5.5 |
| (+-)-(3Z)-5-methyl-3-heptanone oxime OR (+-)-(3E)-5-methyl-3-heptanone oxime | 3-propylphenol | 5.5 |
| (+-)-(3Z)-5-methyl-3-heptanone oxime OR (+-)-(3E)-5-methyl-3-heptanone oxime | 2-isopropyl-5-methylphenol | >5.5 |
| 5-isopropyl-2-methylphenol | 3-propylphenol | >5.5 |
| 5-isopropyl-2-methylphenol | 2-isopropyl-5-methylphenol | >5.5 |
| 3-propylphenol | 2-isopropyl-5-methylphenol | >5.5 |
| 2-ethyl-1-hexanol | (+-)-3-methyl-5-phenyl-1-pentanol | 2 |
| 2-ethyl-1-hexanol | (2E,6Z)-2,6-nonadien-1-ol | 3.1 |
| 2-ethyl-1-hexanol | (+-)-(3Z)-5-methyl-3-heptanone oxime OR (+-)-(3E)-5-methyl-3-heptanone oxime | 4 |
| 2-ethyl-1-hexanol | 5-isopropyl-2-methylphenol | >5.5 |
| 2-ethyl-1-hexanol | 3-propylphenol | >5.5 |
| 2-ethyl-1-hexanol | 2-isopropyl-5-methylphenol | >5.5 |

TABLE 8

Compositions of complex mixtures of antimicrobial perfume ingredient

| Perfume ingredients | Mixture A (% wt) | Mixture B (% wt) | Mixture C (% wt) |
|---|---|---|---|
| 2-ethyl-1-hexanol | 30 | / | 25 |
| 5-isopropyl-2-methylphenol | / | / | 15 |
| (E)-3,7-dimethyl-2,6-octadien-1-ol | / | / | 40 |
| (+-)-3-methyl-5-phenyl-1-pentanol | 30 | 30 | / |
| (Z)-3,7-dimethyl-2,6-octadien-1-ol | 30 | 25 | / |
| (-)-(S)-1,8-P-menthadien-7-ol | / | 20 | / |
| (E)-3-Phenyl-2-propenal | / | 25 | / |
| (+-)-(3Z)-5-methyl-3-heptanone oxime OR (+-)-(3E)-5-methyl-3-heptanone oxime | 10 | / | / |
| 2-isopropyl-5-methylphenol | / | / | 20 |

TABLE 9

Compositions of eight model surfactant bases

| | Ingredients | Model surfactant bases (% wt) | | | | | | | |
|---|---|---|---|---|---|---|---|---|---|
| | | 1 | 2 | 3 | 4 | 5 | 6 | 7 | 8 |
| sodium lauryl ether sulfate (SLES) | SLES 1) | 1.875 | 2.25 | 1.5 | 2.25 | 3.375 | 3.75 | 4.125 | 4.5 |
| Cocamidopropyl betaine (CAPB) | CAPB 2) | 0.625 | 0.75 | 0.75 | 0.75 | 1.125 | 1.25 | 1.375 | 1.5 |
| Coco-glucoside | Coco-glucoside 3) | / | / | 0.75 | / | / | / | / | / |
| Sodium xylene sulfonate | Sodium xylene sulfonate 4) | / | / | / | 3 | / | / | / | / |
| Total surfactant concentration | | 2.5 | 3.0 | 3.0 | 3.0 | 4.5 | 5.0 | 5.5 | 6.0 |
| PERFUME INGREDIENT | | 0.5 | 0.5 | 0.5 | 0.5 | 1 | 1 | 1 | 1 |
| DI water | | Q.S. | Q.S. | Q.S. | Q.S. | Q.S. | Q.S. | Q.S. | Q.S. |
| Total | | 100 | 100 | 100 | 100 | 100 | 100 | 100 | 100 |

1) Texapon® N70 (BASF), 70% surfactant active material
2) TEGO® Betaine F 50 (Evonik Nutrition & Care GmbH), 38% surfactant active material
3) Plantacare® 818 UP (BASF), 52% surfactant active material
4) Sigma Aldrich

TABLE 10

Antimicrobial activities of the complex mixtures of perfume ingredients in model surfactant bases 5-8

| Sample | Log reduction (Log CFU/mL) | | | |
|---|---|---|---|---|
| | Base 5 | Base 6 | Base 7 | Base 8 |
| A | >5.5 | 3 | 1.5 | NT |
| C | >5.5 | >5.5 | >5.5 | 1.2 |
| Control (Base only) | 0 | 0 | 0 | 0 |

NT: not tested

### Conclusions

1. Perfume ingredient that had higher antimicrobial efficacy in ethanol solution had higher antimicrobial activities in the model surfactant base.

2. For Perfume ingredient with high antimicrobial efficacy in ethanol solution, the Perfume ingredient with EACN values of less than or equal to -20) and log P values of less than or equal to 3) had much higher antimicrobial activities in surfactant bases than Perfume ingredient with EACN values greater than -10 or log P values greater than 3.

3. The exceptions were (E)-3-Phenyl-2-propenal and (+-)-(3Z)-5-methyl-3-heptanone oxime OR (+-)-(3E)-5-methyl-3-heptanone oxime, although they have a medium bactericidal activity in ethanol solution and EACN value between -10 and -20, they still had high antimicrobial activity with log reduction greater than 3 in surfactant base as shown in Table 5.

4. Incorporation of hydrotropes and coco-glucoside improved the antimicrobial efficacy of Perfume ingredient in surfactant bases.

Additional perfume ingredients with high bactericidal activity, when tested according to the test procedure as described in Example 1, were listed here: 1-octanol, 1,3,3-trimethylbicyclo[2.2.1]heptan-2-ol, (Z)-2-nonenal, 1-(5-propyl-1,3-benzodioxol-2-yl)ethenone, basil oil, ethyl benzoate, 1,7,7-trimethylbicyclo[2.2.1]heptan-2-ol, benzyl butanoate, 1-butoxycarbonylethyl butanoate, ethyl hexanoate, 4,8-cyclododecadien-1-one, methyl (2E)-2-methyl-2-hexenoate, methyl (E)-3-phenyl-2-propenoate, 3,5,5-trimethyl-1-hexanol, (Z)-4-decenal, 2,6-dimethyl-7-octen-4-one, 2,6-dimethyl-4-heptanol, 1,2-dimethoxy-4-[1-propen-1-yl]benzene, (4E)-4-methyl-5-(4-methylphenyl)-4-pentenal, 1-(3-methyl-1-benzofuran-2-yl)ethenone, (2E,6Z)-2,6-nonadienal, ethyl 2-hydroxybenzoate, 3-methylindole, 8-mercapto-3-p-menthanone, 1,3,3-trimethylbicyclo[2.2.1]heptan-2-one, 1-isopropyl-4-methylbicyclo[3.1.0]hexan-3-one.

The additional perfume ingredients with medium bactericidal activity, when tested according to the test procedure as described in Example 1, were listed here: 2-phenylethyl acetate, methyl 4-methoxybenzoate, 1,3-nonanediyl diacetate, tetrahydro-3-pentyl-4(2H)-pyranyl acetate, 2-furanmethanethiol, 7-isopropyl-2H,4H-1,5-benzodioxepin-3-one, (Z)-3-hexenyl acetate, methyl cyclopentylideneacetate, ethyl 4,6,6-trimethyl-1,3-cyclohexadiene-1-carboxylate, 3-(2,2-dimethylpropyl)pyridine, (Z)-7-decen-4-olide, clove oil, isobutyl isobutyrate, 3,5,6-trimethyl-3-cyclohexene-1-carbaldehyde, 2,4,6-trimethyl-3-cyclohexene-1-carbaldehyde, 2,4-dimethyl-4,4a,5,9b-tetrahydroindeno[1,2-d][1,3]dioxine, spearmint oil, 3-methylbutyl propionate, 2-methylbutyl propionate, 2-isobutyl-3-methoxypyrazine, 2-isobutyl-6-methoxypyrazine, 1-oxo-1-(2-propanyloxy)-2-propanyl 2,2-dimethylpropanoate, methyl 2-hydroxybenzoate, 1-methoxy-3-hexanethiol, 3-phenylbutanal.

We also found the perfume ingredients with high antimicrobial activity in model base 1 are belong to primary alcohols, phenols, aldehydes or oxime, as shown in the table below.

| Oil Name | Functional group | Log reduction in Base 1 (Log CFU/mL) |
|---|---|---|
| (2E)-2-methyl-3-phenyl-2-propenal | Aldehyde | 3.7 |
| (2E,6Z)-2,6-nonadien-1-ol | Primary alcohol | >5.5 |
| 5-isopropyl-2-methylphenol | Phenol | 5.3 |
| (E)-3,7-dimethyl-2,6-octadien-1-ol | Primary alcohol | >5.5 |
| 2-ethyl-1-hexanol | Primary alcohol | 3.6 |
| (Z)-3,7-dimethyl-2,6-octadien-1-ol | Primary alcohol | >5.5 |
| (-)-(S)-1,8-P-menthadiene-7-ol | Primary alcohol | 4.2 |
| (+-)-3-methyl-5-phenyl-1-pentanol | Primary alcohol | 4.3 |
| 2-isopropyl-5-methylphenol | Phenol | >5.5 |
| 3-propylphenol | Phenol | >5.5 |
| 5-methyl-3-heptanone oxime | Oxime | 3.3 |
| (E)-3-Phenyl-2-propenal | Aldehyde | 5.1 |

### Example 5 Effect of Hydrotropes and Solvents in the Formulations According to the Invention

As liquid hand soap usually contains higher surfactant content than the model surfactant bases used in Example 4, therefore, as an example of in vitro efficacy in liquid hand soap bases, model liquid hand soap bases were formulated by increasing the amount of total surfactant and adding commonly used ingredients including EDTA, propylene glycol (PG), glycerol and preservative Nipaguard^{®} CG. The effect of different hydrotropes or solvents on the antimicrobial activity of the formulations was tested using the same method as described in Example 4. All the formulations have a pH of 5-6, adjusted by citric acid and sodium citrate. Results are shown in the tables below.

TABLE 11

Effect of sodium xylene sulfonate on the antimicrobial activity of the formulations according to the invention

| Ingredients (%) | Ex.1 | Ex.2 | Ex.3 | Ex.4 | Ex.5 | Ex.6 | Ex.7 | Ex.8 | Ex.9 | Ex. 10 | Ex. 11 | Ex. 12 |
|---|---|---|---|---|---|---|---|---|---|---|---|---|
| SLES^{a} | 6.00 | 6.00 | 6.00 | 6.00 | 6.75 | 6.75 | 6.75 | 7.50 | 7.50 | 6.00 | 6.75 | 7.50 |
| CAPB^{b} | 2.00 | 2.00 | 2.00 | 2.00 | 2.25 | 2.25 | 2.25 | 2.50 | 2.50 | 2.00 | 2.25 | 2.50 |
| Mixture A | 1.00 | 1.00 | 1.00 | 1.00 | 1.00 | 1.00 | 1.00 | 1.00 | 1.00 | -- | -- | -- |
| Glycerol | 1.00 | 1.00 | 1.00 | 1.00 | 1.00 | 1.00 | 1.00 | 1.00 | 1.00 | 1.00 | 1.00 | 1.00 |
| PG | 1.00 | 1.00 | 1.00 | 1.00 | 1.00 | 1.00 | 1.00 | 1.00 | 1.00 | 1.00 | 1.00 | 1.00 |
| NaCl | 1.00 | 1.00 | 1.00 | 1.00 | 1.00 | 1.00 | 1.00 | 1.00 | 1.00 | 1.00 | 1.00 | 1.00 |
| Nipaguard® CG | 0.01 | 0.01 | 0.01 | 0.01 | 0.01 | 0.01 | 0.01 | 0.01 | 0.01 | 0.01 | 0.01 | 0.01 |
| EDTA.2H2O | 0.10 | 0.10 | 0.10 | 0.10 | 0.10 | 0.10 | 0.10 | 0.10 | 0.10 | 0.10 | 0.10 | 0.10 |
| Citric acid | 0.07 | 0.07 | 0.07 | 0.07 | 0.07 | 0.07 | 0.07 | 0.07 | 0.07 | 0.07 | 0.07 | 0.07 |
| Sodium citrate | 0.42 | 0.42 | 0.42 | 0.42 | 0.42 | 0.42 | 0.42 | 0.42 | 0.42 | 0.42 | 0.42 | 0.42 |
| Sodium xylene sulfonate | -- | 2.50 | 3.00 | 3.50 | 2.50 | 3.00 | 3.50 | 3.50 | 5.00 | 3.50 | 3.50 | 3.50 |
| Log reduction (CFU/mL) | 0.0 | 1.3 | 1.3 | 2.1 | 0.9 | 1.0 | 1.8 | 1.0 | 3.3 | -0.3 | -0.2 | 0.4 |

TABLE 12

Effect of sodium cumene sulfonate on the antimicrobial activity of the formulations according to the invention

| Ingredients (%) | Ex. 13 | Ex.14 | Ex. 15 | Ex.16 | Ex. 17 | Ex.18 | Ex. 19 | Ex.20 | Ex.21 |
|---|---|---|---|---|---|---|---|---|---|
| SLES | 6.00 | 6.00 | 6.00 | 6.75 | 6.75 | 7.50 | 6.00 | 6.75 | 7.50 |
| CAPB | 2.00 | 2.00 | 2.00 | 2.25 | 2.25 | 2.50 | 2.00 | 2.25 | 2.50 |
| Mixture A | 1.00 | 1.00 | 1.00 | 1.00 | 1.00 | 1.00 | -- | -- | -- |
| Glycerol | 1.00 | 1.00 | 1.00 | 1.00 | 1.00 | 1.00 | 1.00 | 1.00 | 1.00 |
| PG | 1.00 | 1.00 | 1.00 | 1.00 | 1.00 | 1.00 | 1.00 | 1.00 | 1.00 |
| NaCl | 1.00 | 1.00 | 1.00 | 1.00 | 1.00 | 1.00 | 1.00 | 1.00 | 1.00 |
| Nipaguard® CG | 0.01 | 0.01 | 0.01 | 0.01 | 0.01 | 0.01 | 0.01 | 0.01 | 0.01 |
| EDTA.2H2O | 0.10 | 0.10 | 0.10 | 0.10 | 0.10 | 0.10 | 0.10 | 0.10 | 0.10 |
| Citric acid | 0.07 | 0.07 | 0.07 | 0.07 | 0.07 | 0.07 | 0.07 | 0.07 | 0.07 |
| Sodium citrate | 0.42 | 0.42 | 0.42 | 0.42 | 0.42 | 0.42 | 0.42 | 0.42 | 0.42 |
| Sodium cumene sulfonate | 2.50 | 3.00 | 3.50 | 2.00 | 2.50 | 2.50 | 3.50 | 3.50 | 3.50 |
| Log reduction (CFU/mL) | 5.0 | >5 | >5 | 2.4 | 2.8 | 2.2 | 0.0 | 0.2 | 0.2 |

TABLE 13

Effect of other hydrotropes or solvents on the antimicrobial activity of the formulations according to the invention

| Ingredients (%) | Ex.22 | Ex.23 | Ex.24 | Ex.25 | Ex.26 | Ex.27 | Ex.28 | Ex.29 | Ex.30 | Ex.31 |
|---|---|---|---|---|---|---|---|---|---|---|
| SLES | 6.00 | 6.75 | 7.50 | 7.50 | 6.00 | 6.75 | 6.00 | 6.75 | 4.50 | 4.50 |
| CAPB | 2.00 | 2.25 | 2.50 | 2.50 | 2.00 | 2.25 | 2.00 | 2.25 | 1.50 | 1.50 |
| Mixture A | 1.00 | 1.00 | 1.00 | -- | 1.00 | 1.00 | -- | -- | 1.00 | 1.00 |
| Glycerol | 1.00 | 1.00 | 1.00 | 1.00 | 1.00 | 1.00 | 1.00 | 1.00 | 1.00 | 1.00 |
| PG | 1.00 | 1.00 | 1.00 | 1.00 | 1.00 | 1.00 | 1.00 | 1.00 | 1.00 | 1.00 |
| NaCl | 1.00 | 1.00 | 1.00 | 1.00 | 1.00 | 1.00 | 1.00 | 1.00 | 1.00 | 1.00 |
| Nipaguard® CG | 0.01 | 0.01 | 0.01 | 0.01 | 0.01 | 0.01 | 0.01 | 0.01 | 0.01 | 0.01 |
| EDTA.2H2O | 0.10 | 0.10 | 0.10 | 0.10 | 0.10 | 0.10 | 0.10 | 0.10 | 0.10 | 0.10 |
| Citric acid | 0.07 | 0.07 | 0.07 | 0.07 | 0.07 | 0.07 | 0.07 | 0.07 | 0.07 | 0.07 |
| Sodium citrate | 0.42 | 0.42 | 0.42 | 0.42 | 0.42 | 0.42 | 0.42 | 0.42 | 0.42 | 0.42 |
| Sodium salicylate | 2.50 | 2.50 | 2.50 | 2.50 | -- | -- | -- | -- | -- | -- |
| Sodium benzoate | -- | -- | -- | -- | 3.50 | 3.50 | 3.50 | 3.50 | -- | -- |
| Isopropylidene glycerol | -- | -- | -- | -- | -- | -- | -- | -- | 2.50 | -- |
| Hexylene glycol | -- | -- | -- | -- | -- | -- | -- | -- | -- | 2.50 |
| Log reduction (CFU/mL) | 3.5 | 3.8 | 2.8 | 0.0 | 1.2 | 0.5 | 0.1 | 0.3 | 3.0 | 3.2 |

As shown in the results, hydrotropes, including sodium xylene sulfonate, sodium cumene sulfonate, sodium salicylate and sodium benzoate, and solvents such as isopropylidene glycerol and hexylene glycol significantly enhanced the antimicrobial effect of perfume ingredients in the formulations. Among the hydrotropes tested, sodium salicylate was the most effective one.

### Example 6 Antimicrobial Efficacy of Formulations With Reduced Level of Perfumery Ingredients

In some products, lower dosage of perfume oil will be applied. Therefore, we also tested the antimicrobial efficacy of model hand soap formulations with reduced content of perfume ingredients. The results are shown in Table 14.

TABLE 14

| Ingredients (%) | Ex.32 | Ex. 33 | Ex. 34 | Ex. 35 | Ex.36 |
|---|---|---|---|---|---|
| SLES | 4.50 | 4.50 | 3.75 | 3.75 | 7.50 |
| CAPB | 1.50 | 1.50 | 1.25 | 1.25 | 2.50 |
| Mixture A | 0.50 | -- | 0.40 | -- | 0.75 |
| Mixture C | -- | 0.50 | -- | 0.40 | -- |
| Glycerol | 1.00 | 1.00 | 1.00 | 1.00 | 1.00 |
| PG | 1.00 | 1.00 | 1.00 | 1.00 | 1.00 |
| NaCl | 1.00 | 1.00 | 1.00 | 1.00 | 1.00 |
| Nipaguard® CG | 0.01 | 0.01 | 0.01 | 0.01 | 0.01 |
| EDTA.2H2O | 0.10 | 0.10 | 0.10 | 0.10 | 0.10 |
| Citric acid | 0.07 | 0.07 | 0.07 | 0.07 | 0.07 |
| Sodium citrate | 0.42 | 0.42 | 0.42 | 0.42 | 0.42 |
| Sodium cumene sulfonate | 3.00 | 3.00 | -- | -- | -- |
| Sodium salicylate | -- | -- | 3.50 | 3.50 | 3.50 |
| Log reduction (CFU/mL) | 2.80 | 3.60 | 3.2 | 2.9 | 2.1 |

Explanation given for the ingredients used in Table 11-14 are summarized in Table 15 below.

TABLE 15

| | |
|---|---|
| Ingredients | |
| SLES | Texapon® N70 (Ex. BASF), 70% surfactant active material |
| CAPB | TEGO® Betaine F 50 (Ex. Evonik Nutrition & Care GmbH), 38% surfactant active material |
| Nipaguard® CG | Preservative blend consisting of methylchloroisothiazolinone and methylisothiazolinone (Ex. |
| | Clariant) |
| Sodium xylene sulfonate | Stepanate®SXS (Ex. Stepan Company), 40% active material |
| Sodium cumene sulfonate | Stepanate®SCS (Ex. Stepan Company), 40% active material |
| isopropylidene glycerol | Augeo® Clean Multi (Ex. Solvay), |
| Hexylene glycol | Ex. Solvay |
| Sodium benzoate | Ex. Sigma-Aldrich |
| Sodium salicylate | Ex. Sigma -Alrich |

### Example 7 In Vitro Efficacy of Mixture A in a Surface Cleaner Product

The anti-bac efficacy of Mixture A in a commercial surface cleaner sample, Pine-Sol® Multi-Surface Cleaner (Lemon Fresh) were determined. In brief, Mixture A were added to Pine-Sol® Multi-Surface Cleaner to final concentrations of 0.25%, 0.5%, 0.75%, 1.0% and 1.25%. Aliquots (120 µL) of each sample were mixed with equal amount of bacterial suspensions of E. coli ATCC 10536 as previously described in wells of 96 well plates, 11 replicates. After contact time of 3 min, viable cells (log CFU) of each well were enumerated. Bactericidal effect, measured as log reduction against control sample of MilliQ water. Table 16 shows Pine-Sol® Multi-Surface Cleaner had no bactericidal effect, in contrast, Mixture A at concentration of 0.25% or greater resulted in greater than 5 log reduction of E. coli after 5 min contact.

TABLE 16

Bactericidal efficacy of Mixture A in Pine-Sol® Multi-Surface Cleaner (Lemon fresh) on E. coli ATCC 10536 after contact time of 5 min

| Samples | Mean log reduction ± SD (n=11) |
|---|---|
| 0.125%Mixture A in 50%Pine-Sol aqueous solution | 3.1 ± 0.3 |
| 0.25% Mixture A in 50%PineSol aqueous solution | 5.3 ± 0.5 |
| 0.375% Mixture A in 50%PineSol aqueous solution | 5.3 ± 0.5 |
| 0.5% Mixture A in 50%PineSol aqueous solution | 5.5 ± 0.3 |
| 0.625% Mixture A in 50%PineSol aqueous solution | 5.3 ± 0.5 |
| 50%Pine-Sol aqueous solution | 0.2 ± 0.3 |

### Example 8 In Vitro Efficacy of the Mixtures Presented Herein in a Soap Bar Base

Table 17 shows that the compositions of soap bar base.

TABLE 17

Compositions of a soap bar base.

| Chemical name | INCI Nomenclature | CAS No. | % |
|---|---|---|---|
| Sodium salt of palm oil fatty acid | Sodium Palmate | 61790-79-2 | 68.6 |
| Sodium salt of palm kernel oil fatty acid | Sodium Palm Kernelate | 61789-89-7 | 17.15 |
| EDTA | Tetrasodium EDTA | 64-02-8 | 0.15 |
| HEDP | Tetrasodium etidronate | 3794-83-0 | 0.10 |
| Sodium chloride | Sodium chloride | 7647-14-5 | 0.5 |
| Glycerol | Glycerol | 56-8'1-5 | 0.5 |
| Water | Aqua | 7732-18-5 | 13 |

Glass bottles (100 mL) containing 25 mL 0.9% saline with a stirring bar were preheated to 60° C. on a magnet stirrer. Soap noodles were grated into a clean glass bottle. Stocks of 25% Mixture A and Mixture C were prepared in dipropylene glycol (DPG). 1 g of grated soap noodle were weighed, and added with 80 mg of stock of Mixture A or Mixture C on a foil. Then soap noodles with mixtures were added to the preheated saline gradually and allow to stir at 300 rpm for 15 min to make test samples of 4% soap suspensions with 0.04% of mixtures. Aliquots (120 µL) of test samples were transferred to a pre-heated 96 well plates, and mixed with equal amount of cell suspension of E. coli ATCC 10536 as described previously. After contact time of 45 s, the viable cells in wells were enumerated. Bactericidal effect, measured as log reduction against control sample of 0.45% saline. Table 18 results show that 0.04% Mixture A and Mixture C had nearly 2 log reduction in 2% soap bar base.

TABLE 18

Bactericidal efficacy of Mixture A & Mixture C in soap bar base on E. coli ATCC 10536 after contact time of 45 s

| Sample | Mean log reduction ± SD (n=11) |
|---|---|
| 2% soap bar base in 0.45% saline | 0.4 ± 0.2 |
| 2% soap bar base with 0.04%Mixture A in 0.45% saline | 1.9 ± 0.3 |
| 2% soap bar base with 0.04%Mixture C in 0.45% saline | 1.8 ± 0.5 |

### Example 9 In Vitro Efficacy of the Mixtures Presented Herein in a Roll-on Deodorant Soap base

Table 19 shows the compositions of Roll-on deodorant soap base.

TABLE 19

Compositions of Roll-on deodorant soap base

| Composition | % |
|---|---|
| Water deionised | 50 |
| 2 NATROSOL 250 H Hydroxyethylcellulose | 0.7 |
| Ethyl alcohol 95° | 40 |
| 1,2-Propylene glycol | 5 |

High antimicrobial efficacy was detected when adding 0.5-1% of the perfumery ingredients according to the invention in the bases.

### Example 10 Synergistic Bactericidal Effect of Mixture C

According to a particular aspect, the compositions provide a synergistic antimicrobial effect; i.e. an effect which is superior to the simple sum or addition of the antimicrobial effect expected when the ingredients of the composition would have been admixed in the desired concentration. In other words, in such cases, antibacterial activity of the combination of combined ingredients is greater than the sum of activities of the individual ingredients.

Referring to the example below, the synergistic effect of the perfume ingredients in the antimicrobial combinations presented herein was discovered by testing a wide range of concentrations (doses) of individual perfume ingredients and mixtures, and observing the bactericidal effect against Escherichia coli ATCC 10536. A determination of the synergistic effect of compositions comprising three or more perfume ingredients was made using the combination index (CI) method for multi-drug systems, described by Chou, T., in Pharmaceutical Reviews 58:621-681 (2006). The CI method determines synergism or antagonism based on the mass-action law principle.

The dose-effect relationship for each drug is described in the below median-effect equation:

$${\text{D=D}}_{\text{m}} {\left[{f}_{\text{a}} / \left(1 - {f}_{\text{a}}\right)\right]}^{\text{l} / \text{m}}$$

Where:
D is a dose that has any given degree of effect (fₐ);
Dₘ is the median-effect dose;
m is the is the coefficient signifying the shape of the dose-effect relationship.

The general equation for the combination index (CI) for n-drug combination at x% effect is as below:

$${}^{\text{n}} {\left(\text{Cl}\right)}_{\text{x}} {\sum }_{j}^{n = 1} \frac{{D}_{j}}{D}$$

Where
D is drug j “alone” that has effect of x%,
Dⱼ is the dose of drug j ‘in combination’ that has effect of x%

The table below denotes the CI values associated with antagonism, additive effects, and synergistic effects.

Description of synergism or antagonism in drug combination studies analyzed with the combination index method

| Range of CI | Description |
|---|---|
| < 0.1 | Very strong synergism |
| 0.1-0.3 | Strong synergism |
| 0.3 - 0.7 | Synergism |
| 0.7-0.85 | Moderate Synergism |
| 0.85 - 0.90 | Slight synergism |
| 0.90-1.10 | Nearly additive |
| 1.10-1.20 | Slight antagonism |
| 1.20 - 1.45 | Moderate antagonism |
| 1.45 - 3.3 | Antagonism |
| 3.3-10 | Strong antagonism |
| > 10 | Very strong antagonism |

Determination of Synergy via the Combination Index (CI): The CI was calculated using Compusyn software (ComboSyn, Inc., as created by Dr. Dorothy Chou in 2005), according to the manufacturer’s instructions. The concentration of each test composition (combinations of more than one perfume ingredient, and comparative compositions comprising the single perfume ingredients used to generate the combination) and their corresponding effect fraction was entered, and the corresponding CI calculated.

When the calculated CI values were less than 0.9, the combination of perfume ingredients was regarded as having synergistic effect. When the calculated CI was between 1.0 a 0.9, the combination of perfume ingredients was regarded as having an additive effect. When the calculated CI was greater than 1.0, the combination of perfume ingredients was regarded as having an antagonistic effect.

Table 20 show the bactericidal dose related effect (log reduction) and calculated effect fraction of the Mixture C and compositions against E. coli ATCC 10536. Stock of compositions were prepared in 100% ethanol, then diluted in MilliQ water to obtain 2 × final concentration in 40% ethanol. BCT tests were conducted on samples against E. coli for a contact time of 45 s.

TABLE 20

Bactericidal dose effect and calculated effect fraction of Mixture C and compositions against E. coli ATCC 10536 after contact time of 45 s

| Sample Name | BCT_45s_E. coli ATCC 10536 in 20% EtOH | | | | | |
|---|---|---|---|---|---|---|
| 5-isopropyl-2-methylphenol | Dose (% w/v) | 0.030 | 0.035 | 0.040 | 0.045 | 0.050 |
| | Log reduction | 0.660 | 1.812 | 4.379 | 5.960 | 6.960 |
| | Effect fraction | 0.090 | 0.250 | 0.610 | 0.830 | 0.970 |
| 2-ethyl-1-hexanol | Dose (% w/v) | 0.060 | 0.065 | 0.070 | 0.075 | 0.080 |
| | Log reduction | 0.367 | 0.728 | 2.052 | 3.678 | 5.760 |
| | Effect fraction | 0.050 | 0.100 | 0.290 | 0.510 | 0.800 |
| (E)-3,7-dimethyl-2,6-octadien-1-ol | Dose (% w/v) | 0.040 | 0.050 | 0.055 | 0.060 | 0.070 |
| | Log reduction | -0.175 | 0.909 | 2.474 | 4.760 | 7.160 |
| | Effect fraction | -0.020 | 0.130 | 0.350 | 0.660 | 1.000 |
| 2-isopropyl-5-methylphenol | Dose (% w/v) | 0.025 | 0.030 | 0.035 | 0.040 | 0.045 |
| | Log reduction | 0.887 | 2.140 | 3.344 | 4.987 | 6.387 |
| | Effect fraction | 0.120 | 0.300 | 0.470 | 0.690 | 0.890 |
| Mixture C | Dose (% w/v) | 0.035 | 0.040 | 0.045 | 0.050 | 0.055 |
| | Log reduction | 0.514 | 1.839 | 3.404 | 5.987 | 6.587 |
| | Effect fraction | 0.070 | 0.260 | 0.470 | 0.830 | 0.920 |

Table 21 show the calculated combination index (CI) value of a range of concentrations of Mixture C. Synergistic bactericidal effect was found for 0.05 and 0.055% Mixture C against E. coli ATCC 10536.

TABLE 21

Combination index values of MIXTURE C at a range of concentrations

| Dose (% w/v) | CI of Mixture C |
|---|---|
| 0.035 | 0.914 |
| 0.040 | 0.900 |
| 0.045 | 0.928 |
| 0.050 | 0.880 |
| 0.055 | 0.895 |

Synergistic: Cl < 0.90 Additive 1.10 ≥ Cl ≥ 0.90

Publications cited throughout this document are hereby incorporated by reference in their entirety. Although the various aspects of the invention have been illustrated above by reference to examples and preferred embodiments, it will be appreciated that the scope of the invention is defined not by the foregoing description but by the following claims properly construed under principles of patent law.

1. An antimicrobial composition comprising:
a. a perfume ingredient having a log P of less than or equal to 3.5, and a bactericidal effect of a 5.5 log reduction in an ethanol solution at a concentration of less than or equal to 0.5% in ethanol solution; and
b. a surfactant.

2-4. (canceled)

5. The antimicrobial composition of claim 1, wherein the perfume ingredient has an equivalent alkane carbon number of less than or equal to -10.

6-7. (canceled)

8. The antimicrobial composition of claim 1, wherein the perfume ingredient is at least 0.001 % (w/v) of the antimicrobial composition.

9-12. (canceled)

13. The antimicrobial composition of claim 1, wherein the surfactant is selected from the group consisting of: an anionic surfactant, a non-ionic surfactant, an amphoteric surfactant and a combination thereof.

14-17. (canceled)

18. The antimicrobial composition of claim 1, wherein the antimicrobial composition further comprises a hydrotrope.

19. (canceled)

20. The antimicrobial composition of claim 1, wherein the antimicrobial composition further comprises an additional agent active against gram-positive or gram-negative bacteria.

21. (canceled)

22. The antimicrobial composition of claim 1, wherein the perfume ingredient is selected from the group consisting of: 1-phenylethyl acetate, (2E)-2-methyl-3-phenyl-2-propenal, (2E,6Z)-2,6-nonadien-1-ol, 5-isopropyl-2-methylphenol, ethyl (E)-3-phenyl-2-propenoate, 3,7-dimethyl-2,6-octadienal, 3-[4-methyl-3-cyclohexen-1-yl]-1-butanol, 4-decanolide, 2-methoxy-4-propylphenol, (E)-3,7-dimethyl-2,6-octadien-1-ol,2,4,6-trimethyl-3-cyclohexene-1-methanol, (2E)-2-methyl-3-(4-methylphenyl)-2-propen-1-ol, 2,5-dimethyl-2-indanmethanol, 2,2-dimethyl-3-[3-methyl-2,4-pentadien-1-yl]oxirane, 2-ethyl-1-hexanol, (Z)-3,7-dimethyl-2,6-octadien-1-ol, (Z)-6-nonen-1-ol, 1,8-p-menthadien-7-ol, 3-methyl-5-phenyl-1-pentanol, 2,6,6-trimethyl-1,3-cyclohexadiene-1-carbaldehyde, 2-isopropyl-5-methylphenol, 3-propylphenol, ethyl 2-hydroxybenzoate, 4-isopropyl-1-benzenemethanol, 7-propyl-2H,4H-1,5-benzodioxepin-3-one, 2-methoxy-4-(2-propen-1-yl)phenol, 3-(1,3-benzodioxol-5-yl)-2-methylpropanal, indole, 2-methoxy-4-[(1E)-1-propen-1-yl]phenol, 3-methyl-2-[(2Z)-2-penten-1-yl]-2-cyclopenten-1 -one, (E)-2-hexenyl acetate, 4-(2-methyl-2-propanyl)cyclohexanone, ethyl phenyl acetate, 5-methyl-3-heptanone oxime, 2-(4-methylcyclohex-3-enyl)propan-2-ol, (E)-3-phenyl-2-propenenitrile, 2-phenyl-1-propanol, (E)-3-phenyl-2-propenal, methyl 2-aminobenzoate, 4-nonanolide, 6-pentyltetrahydro-2H-pyran-2-one, 2-methyl-4-phenyl-2-butanol, 2-methyl-1-phenyl-2-propanol, 2-phenylethyl formate, (E)-2-hexen-1-ol, 4-methylphenol, 4,4a,6,7,8,8a-hexahydro-1,4-methanonaphthalen-5(1H)-one, 1-oxo-1-(2-propanyloxy)-2-propanyl 2,2-dimethylpropanoate, 2,4,6-trimethyl-3-cyclohexene-1-carbaldehyde, 3,5,6-trimethyl-3-cyclohexene-1-carbaldehyde, 3-(4-hydroxy-4-methylpentyl)-3-cyclohexene-1-carbaldehyde, 4-(4-hydroxy-4-methylpentyl)-3-cyclohexene-1-carbaldehyde, (2E)-3-phenyl-2-propen-1-ol, 2-furanmethanethiol, (Z)-3-hexenyl formate, 2-phenylethyl acetate, 1-octanol, 1,3,3-trimethylbicyclo[2.2.1]heptan-2-ol, (Z)-2-nonenal, methyl 4-methoxybenzoate, 1,3-nonanediyl diacetate, tetrahydro-3-pentyl-4(2H)-pyranyl acetate, 1-(5-propyl-1,3-benzodioxol-2-yl)ethenone, ethyl benzoate, 1,7,7-trimethylbicyclo[2.2.1]heptan-2-ol, benzyl butanoate, 1-butoxycarbonylethyl butanoate, ethyl hexanoate, 4,8-cyclododecadien-1-one, methyl (2E)-2-methyl-2-hexenoate, methyl (E)-3-phenyl-2-propenoate, (Z)-3-hexenyl acetate, 3,5,5-trimethyl-1-hexanol, methyl cyclopentylideneacetate, (Z)-4-decenal, 2,6-dimethyl-7-octen-4-one, 2,6-dimethyl-4-heptanol, 3-(2,2-dimethylpropyl)pyridine, (Z)-7-decen-4-olide, clove oil, isobutyl isobutyrate, 2,4-dimethyl-4,4a,5,9b-tetrahydroindeno[1,2-d][1,3]dioxine, 1,2-dimethoxy-4-[1-propen-1-yl]benzene, 1-(3-methyl-1-benzofuran-2-yl)ethenone, (2E,6Z)-2,6-nonadienal, 3-methylbutyl propionate, 2-methylbutyl propionate, 2-isobutyl-3-methoxypyrazine, 2-isobutyl-6-methoxypyrazine, methyl 2-hydroxybenzoate, 3-methylindole, 1-methoxy-3-hexanethiol, 8-mercapto-3-p-menthanone, 1,3,3-trimethylbicyclo[2.2.1]heptan-2-one, 1-isopropyl-4-methylbicyclo[3.1.0]hexan-3-one, 3-phenylbutanal, 6-hexyltetrahydro-2H-pyran-2-one, 7-isopropyl-2H,4H-1,5-benzodioxepin-3-one, spearmint oil and a combination thereof.

23-28. (canceled)

29. The antimicrobial composition of claim 1, wherein the perfume ingredient is an aldehyde, a primary alcohol, a phenol or an oxime.

30-38. (canceled)

39. The antimicrobial composition according to claim 1 further comprising a solvent.

40-42. (canceled)

43. The antimicrobial compostion according to claim 1 further comprising ingredients such as colorants, preservatives, viscosifiers, opacifiers, emollients, humectants, antioxidants, gelling agents, gums, chelators, functional polymers, cellulose derivatives, essential oils, electrolytes, and pH adjusters.

44. (canceled)

45. The antimicrobial compostion of claim 1 comprising a mixture of 2-ethyl-1-hexanol, 5-isopropyl-2-methylphenol, (E)-3,7-dimethyl-2,6-octadien-1-ol, and 2-isopropyl-5-methylphenol.

46. (canceled)

47. The antimicrobial compostion of claim 1 comprising a mixture of (E)-3-phenyl-2-propenal; (Z)-3,7-dimethyl-2,6-octadien-1-ol; 1,8-p-methadien-7-ol; and 3-methyl-5-phenyl-1-pentanol.

48. (canceled)

49.The antimicrobial compostion of claim 1 comprising a mixture of 2-ethyl-1-hexanol; (Z)-3,7-dimethyl-2,6-octadien-1-ol; 3-methyl-5-phenyl-1-pentanol; and 5-methyl-3-heptanone oxime.

50. (canceled)

51.The antimicrobial compostion of claim 1 comprising a mixture of, 5-isopropyl-2-methylphenol, (E)-3,7-dimethyl-2,6-octadien-1-ol, and 2-isopropyl-5-methylphenol.

52. (canceled)

53. The antimicrobial composition of claim 1, further comprising a malodor neutralizing system.

54. (canceled)

55. The antimicrobial composition of claim 1, further comprising a malodor antagonist system.

56-57. (canceled)

58. A consumer product comprising the antimicrobial composition according to claim 1, wherein the consumer product is selected from the group consisting of: a hair care product, a body care product, a skin care product, an oral care product, a female care product, a home care product, a laundry care product, or a body cleansing product, including but not limited to shampoo, shower gel, facial cleanser, shaving gel, liquid hand soap, foaming soap, hand sanitizer, soap bar, mouthwash, toothpaste, female hygiene composition, fabric cleanser, carpet cleanser, all purpose cleanser, dishwashing detergent, fresh produce detergent, deodorant, air fresher and air disinfectant.

59. A method for eliminating or/and reducing the number of microbes on a surface or body part comprising contacting the surface or body part with the antimicrobial composition of claim 1.

60. (canceled)
